(19) United States
(12) Patent Application Publication
(10) Pub. No.: US 2014/0113258 A1
(43) Pub. Date: Apr. 24, 2014

(54) Electronic Hand Assessment Tool and Method of Using the Same

(71) Applicant: ELIZABETHTOWN COLLEGE, Elizabethtown, PA (US)

(72) Inventors: Kurt M. DeGoede, Mount Joy, PA (US); Daniel Panchik, Hershey, PA (US)

(73) Assignee: ELIZABETHTOWN COLLEGE, Elizabethtown, PA (US)

(21) Appl. No.: 14/058,904

(22) Filed: Oct. 21, 2013

Related U.S. Application Data
(60) Provisional application No. 61/716,525, filed on Oct. 20, 2012.

(51) Int. Cl. A61B 5/11

(52) U.S. Cl. CPC A61B 5/11
USPC 434/155

(57) ABSTRACT

The present invention relates to a dual-purpose assessment and intervention tool that uses motion capture technology to measure fine motor control through the task of handwriting. The intervention portion of the tool contains various subsections designed to measure fine motor control, including tracing a maze and both tracing and copying a variety of characters. The assessment portion of the tool contains a standard set of exercises designed to provide an overall impression of fine motor control. The assessment portion of the tool generates a numerical score based on pixel-by-pixel accuracy and speed of writing.

### CROSS-REFERENCE TO RELATED APPLICATIONS

This application claims the benefit of priority to U.S. Provisional Application Ser. No. 61/716,525, filed Oct. 20, 2012, the entire disclosure of which is incorporated by reference herein in its entirety.

### BACKGROUND OF THE INVENTION

The ability to use the hands in a functional manner is of great importance to an individual. For example, it is well accepted that the use of one's hands has a direct impact on one's well-being and health. Therefore, when an individual suffers an injury or impairment to the hand and/or wrist, it is important to help that individual gain or regain as much function as possible.

Hand and wrist impairments can be a devastating occurrence for both adults and children, particularly because they impact the performance of everyday tasks. Hand and wrist impairments affect hand skills, which, in turn, affect a person's performance and engagement in activities. Handwriting is one of the activities disrupted by a hand or wrist impairment, so occupational therapists often use the task of handwriting to measure performance of hand skills in their clients. Currently, there are a limited number of objective assessment tools to measure fine motor control of the hand.

Thus, a need exists for an objective assessment tool that may be useful for treating fine motor problems due to hand and wrist impairments in patients across the developmental spectrum. The present invention satisfies this need.

### SUMMARY OF THE INVENTION

The present invention relates to an electronic hand assessment tool. The tool includes a computing device having a visual display, a stylus, and a software application executable on the computing device, such that the software application presents a graphical user interface on the visual display, and the software application presents at least one handwriting exercise to be performed by a subject contacting the stylus to the visual display. In one embodiment, the at least one handwriting exercise is selected from the group consisting of a trace, a copy and a maze. In another embodiment, the tool further includes a scoring system for scoring the performance of the at least one handwriting exercise. In another embodiment, the scoring system is based on pixel-by-pixel accuracy. In another embodiment, the scoring system is based on speed of writing. In another embodiment, the scoring system is based on speed of writing and pixel-by-pixel accuracy. In another embodiment, the scoring system compares pixels between a target and a user drawn image. In another embodiment, a score value is awarded on a graded continuum that allots full value points for marks of the user drawn image overlapping the target and a lower score for stray marks of the user drawn image with progressive decrease in score with increasing distance of marks of the user drawn image from the target. In another embodiment, the scoring system is based on a pixel overlap ratio. In another embodiment, a score value is based on the amount of the target that is overlapped, how much of the user drawn image is written outside of the lines of the target, and the percentage of how much of the target is colored in by the user drawn image. In another embodiment, the scoring system comprises a plurality of difficulty settings. In another embodiment, the tool further includes an assessment mode. In another embodiment, the tool further includes an intervention mode.

The present invention also relates to a method of measuring fine motor control. The method includes the steps of obtaining at least one electronic handwriting exercise on a visual display of a computing device, performing the at least one electronic handwriting exercise by contacting a stylus to the visual display, and measuring fine motor control based on the performance of the at least one handwriting exercise. In one embodiment, the at least one handwriting exercise is selected from the group consisting of a trace, a copy and a maze. In another embodiment, the method includes the step of scoring the performance of the at least one handwriting exercise. In another embodiment, the scoring is based on pixel-by-pixel accuracy. In another embodiment, the scoring is based on speed of writing. In another embodiment, the scoring is based on speed of writing and pixel-by-pixel accuracy. In another embodiment, the method includes the step of comparing pixels between a target and a user drawn image.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a screenshot of an exemplary menu of selectable parameters.

FIG. 2 is a screenshot an exemplary trace handwriting exercise.

FIG. 3 is a screenshot an exemplary trace handwriting exercise.

FIG. 4 is a screenshot an exemplary trace handwriting exercise.

FIG. 5 is a screenshot an exemplary copy handwriting exercise.

FIG. 6 is a screenshot an exemplary copy handwriting exercise.

FIG. 7 is a screenshot an exemplary maze handwriting exercise.

FIG. 8 is a screenshot an exemplary maze handwriting exercise.

FIG. 9 is a screenshot of an exemplary menu of selectable parameters.

FIG. 10 is a screenshot of an exemplary menu of selectable parameters.

FIG. 11 is a screenshot an exemplary maze handwriting exercise.

FIG. 12 is a screenshot an exemplary trace handwriting exercise.

FIG. 13 is a screenshot an exemplary copy handwriting exercise.

FIG. 14 is an exemplary data summary chart of a user session.

FIG. 15 is an exemplary data summary chart of a user session.

FIG. 16 is a graph of the relationship between E-HAT and Beery VMI-Full Score.

FIG. 17 is a graph of the relationship between E-HAT and Beery VMI-Coordination Score.

FIG. 18 is a graph of the relationship between Initial E-HAT and Immediate Retest Scores.

FIG. 19 is a graph of the relationship between Initial E-HAT and Delayed Retest Scores.

FIG. 20 is a screenshot of an exemplary menu of selectable parameters.

FIG. 21 is a set of screenshots of exemplary Trace Subtest (A), a Copy Subtest (B) and a Copy/Disappear Subtest (C).

FIG. 22 is a photograph of an exemplary tablet PC computing device running the E-HAT application platform.

FIG. 23 is a comparison chart of improvements in accuracy by subtests (A) and in speed by subtests (B).

FIG. 24 is a graph of the relationship between E-HAT and Beery VMI-Full Score.

### DETAILED DESCRIPTION

It is to be understood that the figures and descriptions of the present invention have been simplified to illustrate elements that are relevant for a clear understanding of the present invention, while eliminating, for the purpose of clarity, many other elements found in hand assessment systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The Electronic Hand Assessment Tool (E-HAT) is a software application designed to run on a computing device having a visual display interface, such as a tablet computer. In other embodiments, E-HAT may run on any computing device understood by those skilled in the art, including laptops, desktops, smartphones game consoles, PDAs and the like, provided the computing device has an electronic visual display capable of touchscreen user interaction and control. The E-HAT application is a dual-purpose assessment and intervention tool that uses motion capture technology to measure fine motor control through the task of handwriting. In one embodiment, an intervention portion of the tool contains various subsections designed to measure fine motor control, including, without limitation, tracing a maze and both tracing and copying a variety of characters. In another embodiment, the assessment portion of the tool contains a standard set of exercises designed to provide an overall impression of fine motor control. For example, the assessment portion of the tool generates a numerical score based on pixel-by-pixel accuracy and speed of writing.

The present invention uses motion capture technology to collect objective, quantitative measurements of fine motor control. The present invention fills a need in occupational therapy for an objective hand function assessment. It also provides therapists with a low-cost option for evaluation and intervention purposes by utilizing the motion capture technology already installed on a tablet computer.

The system of the present invention includes a scoring metric, or algorithm, by which to weight each information item category in the system, and to calculate a value. It should be appreciated that the values designated for each information item category may vary. Further, the number or combination of information item categories will also effect the values designated. It should be appreciated that the system of the present invention is not limited to any predetermined value, number or other nomenclature.

For example, an objective score is obtained after each character is completed based on speed of writing and pixel-by-pixel accuracy. In one embodiment, the scoring algorithm compares pixels between the target (known as the letter) and the user drawn image (known as the trace). In the scoring summary, the percent of pixel overlap is the percent of overlap between the pixels of the letter and the trace. Points are awarded on a graded continuum that allots full points for marks overlapping the target and a lower score for stray marks with progressive decrease in score with increasing distance from the target. Scoring data may be stored under a separate file for future reference. In another embodiment, the scoring algorithm is based on a pixel overlap ratio. This examines the amount of the original letter that is overlapped, how much is written outside of the lines of the original letter, and the percentage of how much of the original letter is colored in.

In one embodiment, the system allows for different scoring “difficulty” settings for therapeutic use, but in assessment mode the scoring is preferred with the “hard” settings. For example, scoring settings may include, without limitation:
name=“Easy”;
exp=1;
lin=0.15;
bonus_pixels=10;
bonus_value=0.75;
name=“Normal”;
exp=1;
lin=0.12;
bonus_pixels=7;
bonus_value=0.5;
name=“Hard”;
exp=1;
lin=0.10;
bonus_pixels=4;
bonus_value=0.25;
break;
   exp->“exponential_factor”>Exponential alterating of the score—not used
   exp->(all scoring sets this value to 1.0)
   lin->“grace_factor”>Linear alteration of the score<
   bonus_pixels“>The amount of pixels outside the letter that are still worth points</param>
   bonus_point_value”>The amount of points that bonus pixels are worth. Values between 0 and 1</param>

In one embodiment, the algorithm scans through all pixels in field. If the letter contains the pixel add 1 to total, if that same pixel is also on in the trace add 1 to sum. If there are trace pixels within bonus_pixels of the letter pixel in question, each of those pixels adds bonus_value to sum. However every pixel in the trace not in the letter also adds 1 to total.

The effect of these last two elements together may be that stray pixels that are close to the letter target pixels do not count against you as much as stray pixels that are further away (but all stray pixels count against your score). In “easy” stray pixels in the trace that are within 10 pixels of the letter are only 25% as damaging to your score as stray pixels further than 10 pixels from the target letter. In “hard” scoring stray pixels within 4 pixels of the target are 75% as damaging to your score as pixels more than 4 pixels from the target letter.

The score is calculated by:

Score=(sum/(total*(1−linear_factor)))̂exponential_factor.

Again recall the exponential factor was included as a possible way to tune the scoring put is not currently being utilized.

The effect of the linear factor is a forgiveness level. In the hard setting you can earn a perfect 100 score with any Score over 90—the first 10% of the target pixels you miss do not count against your score. In the easy setting it is the first 15% not counted against you. Any score over 100 is reported as 100.

The raw % pixel overlap is also reported with the score.

It should be appreciated that other scoring metrics and value weightings may be used, without limitation, provided the scoring algorithm provides for scaled scoring output suitable for both assessment and training of a user of the present invention.

Generally, the system of the present invention may operate on a computer platform, such as a local or remote executable software platform, or as a hosted internet or network program or portal. In certain embodiments, only portions of the system may be computer operated, or in other embodiments, the entire system may be computer operated. As contemplated herein, any “computer platform” may include any computing device as would be understood by those skilled in the art, including desktop or mobile devices, laptops, desktops, tablets, smartphones or other wireless digital/cellular phones, televisions or other thin client devices.

For example, the computer operable component(s) of the system may reside entirely on a single computing device, or may reside on a central server and run on any number of end-user devices via communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data format between two or more computing devices. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

Further, the communications network may also use standard architecture and protocols as understood by those skilled in the art, such as, for example, a packet switched network for transporting information and packets in accordance with a standard transmission control protocol/Internet protocol (“TCP/IP”). Any of the computing devices may be communicatively connected into the communications network through, for example, a traditional telephone service connection using a conventional modem, an integrated services digital network (“ISDN”), a cable connection including a data over cable system interface specification (“DOCSIS”) cable modem, a digital subscriber line (“DSL”), a T1 line, or any other mechanism as understood by those skilled in the art. Additionally, the system may utilize any conventional operating platform or combination of platforms (Windows, Mac OS, Unix, Linux, Android, etc.) and may utilize any conventional networking and communications software as would be understood by those skilled in the art.

Further, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. For example, a system administrator may allow for administration at one or more levels, such as at an individual user (patient) level, a healthcare professional level, or at a system level. A system administrator may also implement access or use restrictions for users at any level. Such restrictions may include, for example, the assignment of user names and passwords that allow the use of the present invention, or the selection of one or more data types that the subservient user is allowed to view or manipulate.

As mentioned previously, the system may operate as application software, which may be managed by a local or remote computing device. The software may include a software framework or architecture that optimizes ease of use of at least one existing software platform, and that may also extend the capabilities of at least one existing software platform. The application architecture may approximate the actual way users organize and manage electronic files, and thus may organize use activities in a natural, coherent manner while delivering use activities through a simple, consistent, and intuitive interface within each application and across applications. The architecture may also be reusable, providing plug-in capability to any number of applications, without extensive re-programming, which may enable parties outside of the system to create components that plug into the architecture. Thus, software or portals in the architecture may be extensible and new software or portals may be created for the architecture by any party.

The system software may provide, for example, applications accessible to one or more users to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system.

The system software may also be a portal that provides, via the GUI, remote access to and from the system of the present invention. The software may include, for example, a network browser, as well as other standard applications. The software may also include the ability, either automatically based upon a user request in another application, or by a user request, to search, or otherwise retrieve particular data from one or more remote points, such as on the internet. The software may vary by user type, or may be available to only a certain user type, depending on the needs of the system. Users may have some portions, or all of the application software resident on a local computing device, or may simply have linking mechanisms, as understood by those skilled in the art, to link a computing device to the software running on a central server via the communications network, for example. As such, any device having, or having access to, the software may be capable of uploading, or downloading, any information item or data collection item, or informational files to be associated with such files.

Presentation of data through the software may be in any sort and number of selectable formats. For example, a multi-layer format may be used, wherein additional information is available by viewing successively lower layers of presented information. Such layers may be made available by the use of drop down menus, tabbed pseudo manila folder files, or other layering techniques understood by those skilled in the art. Formats may also include AutoFill functionality, wherein data may be filled responsively to the entry of partial data in a particular field by the user. All formats may be in standard readable formats, such as XML. The software may further incorporate standard features typically found in applications, such as, for example, a front or “main” page to present a user with various selectable options for use or organization of information item collection fields.

The system software may also include standard reporting mechanisms, such as generating a printable results report, or an electronic results report that can be transmitted to any communicatively connected computing device, such as a generated email message or file attachment. Likewise, particular results of the system can trigger an alert signal, such as the generation of an alert email, text or phone call, to alert an expert, clinician or other healthcare professional of the particular results.

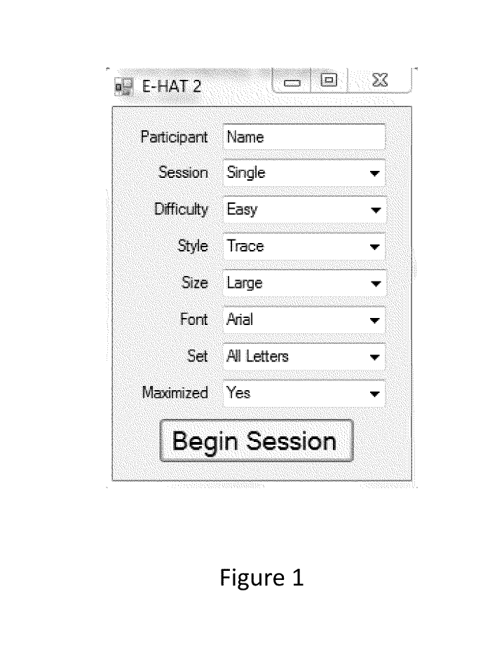

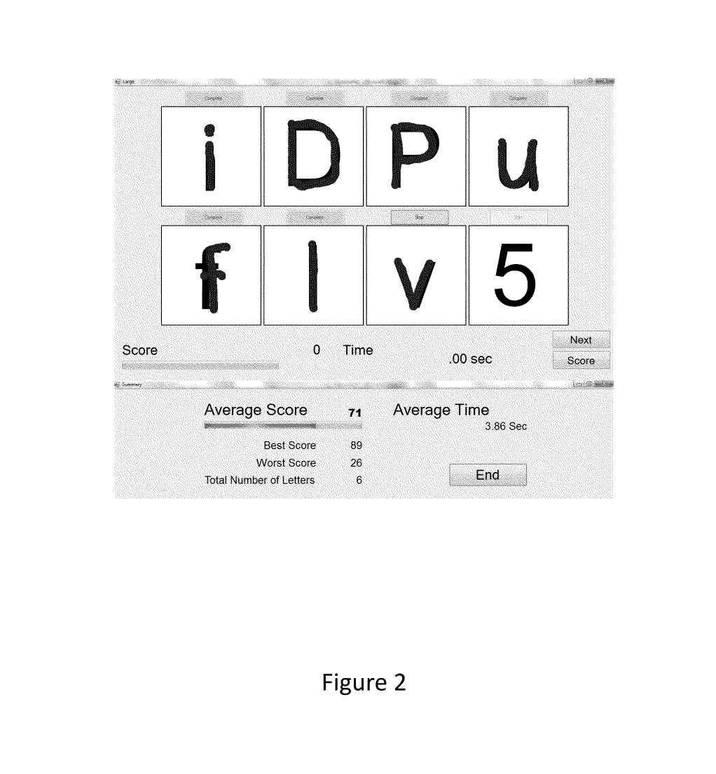

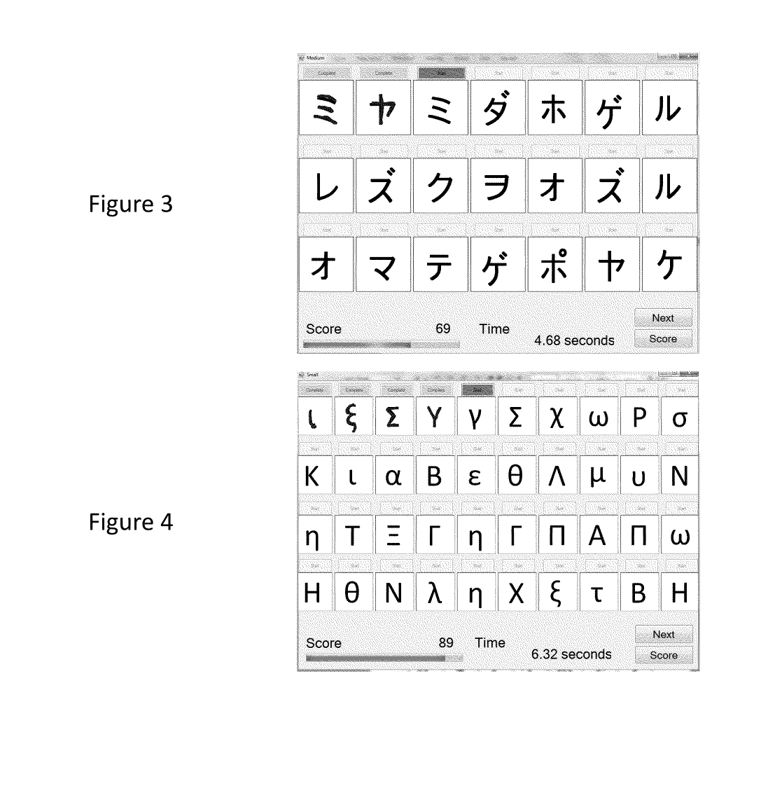

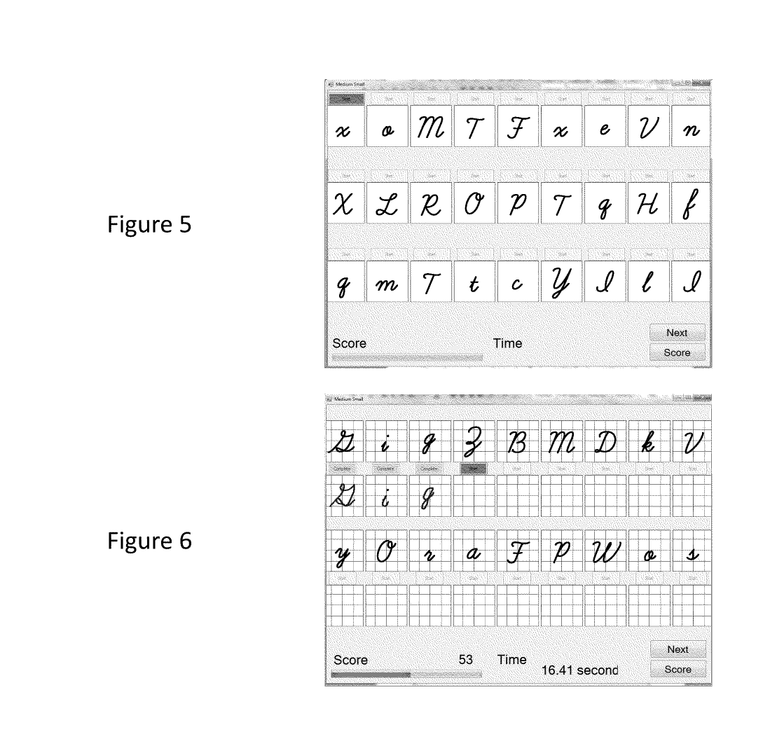

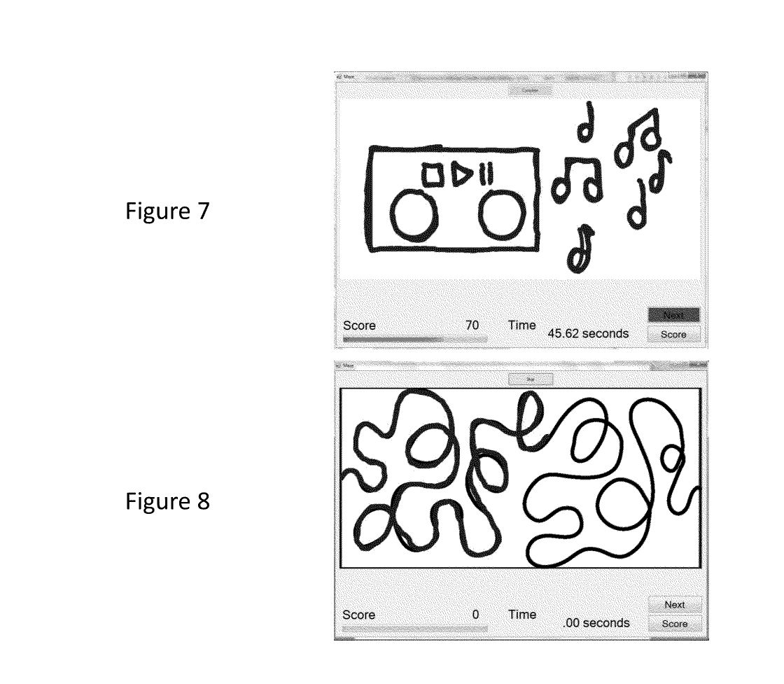

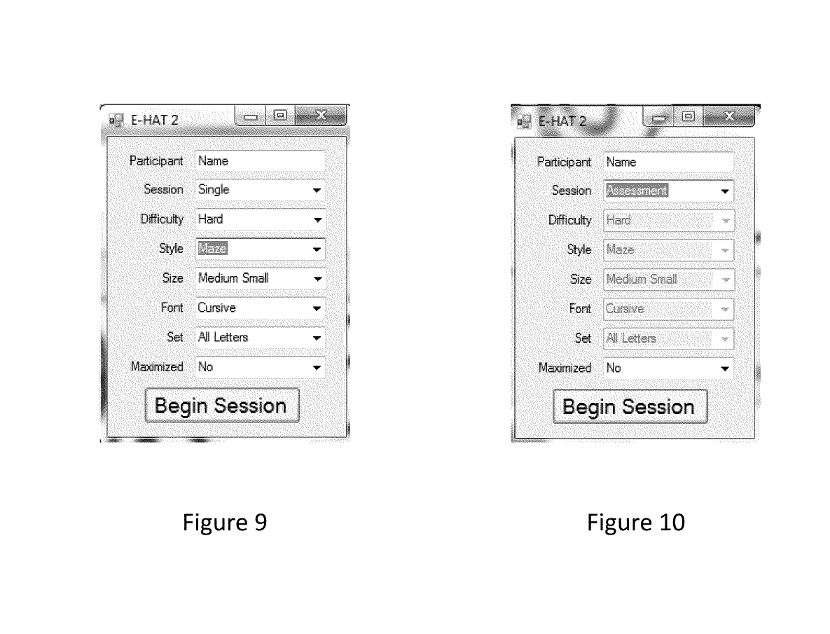

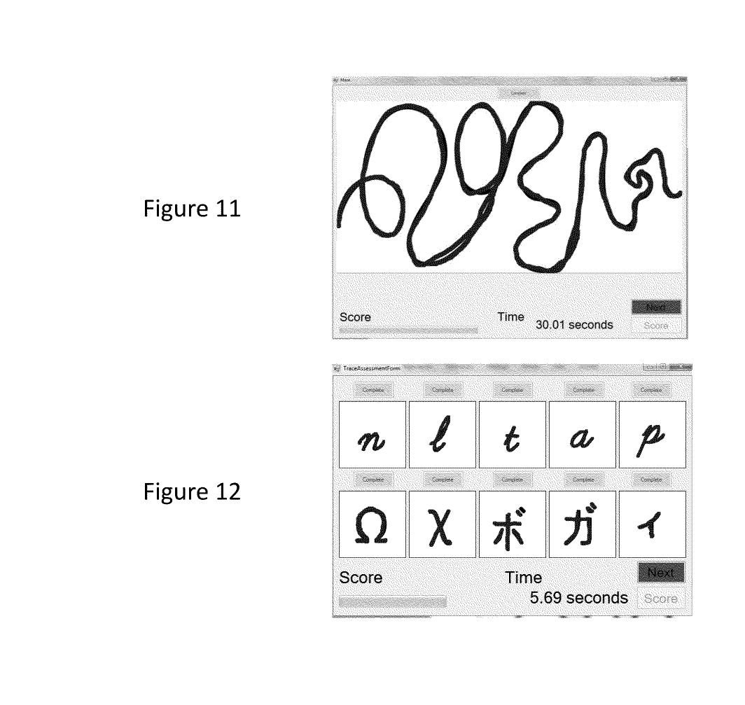

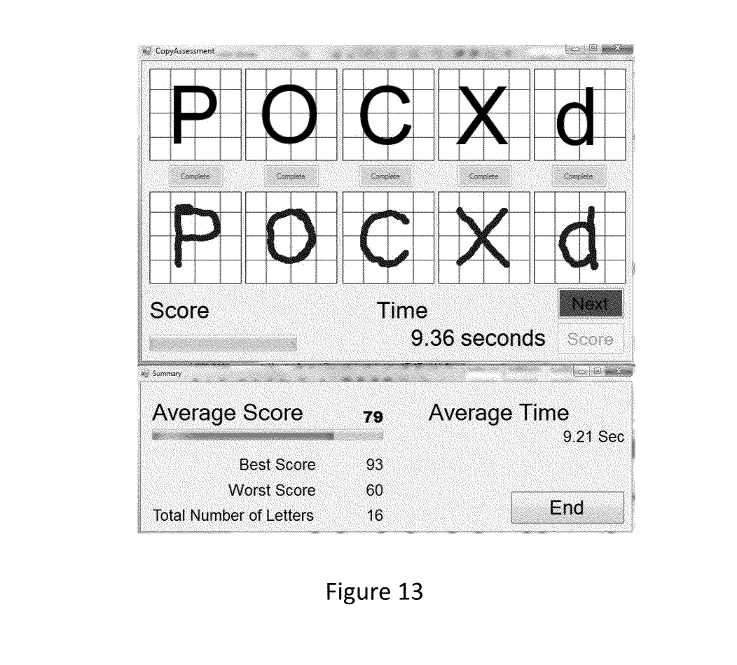

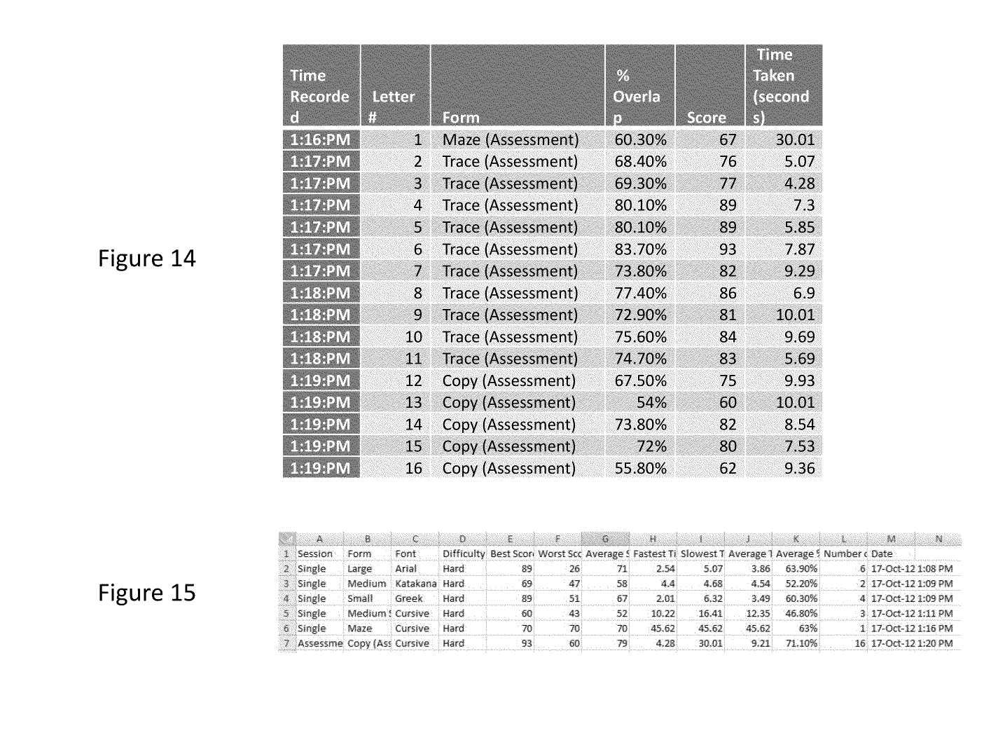

FIGS. 1-15 are exemplary screenshots and data output of the E-HAT platform. For example, FIGS. 1, 9 and 10 show exemplary customizable menus, allowing a user to select the desired parameters to begin a user session. FIGS. 2-4, 12 are exemplary trace handwriting exercises, illustrating the target letters and at least partial user drawn trace. The system may include any number of handwriting exercises in total, and any number of handwriting exercises per page. The system also may illustrate individual scores, average scores, individual times and average times, and the like. Shown in FIGS. 5, 6 and 13 are exemplary copy handwriting exercises, illustrating the target letters to copy and a blank space for the user to copy the target letter. As with the trace exercises, the system may include any number of copy handwriting exercises in total, and any number of handwriting exercises per page. The system also may illustrate individual scores, average scores, individual times and average times, and the like. Shown in FIGS. 7, 8 and 11 are exemplary maze handwriting exercises, illustrating the target patterns to copy and and at least partial user drawn trace. As with the trace exercises, the system may include any number of maze handwriting exercises in total, and any number of handwriting exercises per page. The system also may illustrate individual scores, average scores, individual times and average times, and the like. FIGS. 14 and 15 show exemplary summary data for the exercises performed. This data can be organized as desired by the user or administrator, and may include information items such as testing time, letter number, form of test, percent overlap or ratio, calculated score, and time taken to complete the exercise. Information items may also include the listing of parameters such as, without limitation, session, form, font, difficulty, best score, worst score, average score, fastest time, slowest time, average time, number of tries, and the date. The system may further generate reports for display, electronic transmission, or printer ready.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

The study used a methodological research design with a quantitative approach to data collection. Methodological research is used to develop the validity and reliability of instruments to measure constructs used in research and clinical practice (Carter, Lubinsky, & Domholdt, 2011; Portney & Watkins, 2008). Establishing the reliability and validity of assessment tools is an important objective of methodological research to ensure that the data collected from these instruments can support effective clinical reasoning (Benson & Schell, 1997; Carter, Lubinsky, & Domholdt, 2011).

### Reliability and Validity Measures

Reliability refers to the consistency of a measurement derived from the assessment, while validity seeks to ensure that the assessment tool is accurately assessing the intended construct (Kielhofner, 2006; Nunally, 1978). Several types of reliability and validity exist and should be taken into consideration when developing an instrument; however, the intended purpose of the instrument impacts which of these measurements are most appropriate (Law, 1987). Evaluative measurement tools, such as the present invention (referred to periodically herein as E-HAT) should focus on reliability measures that match the intended format, and the validity measures should focus on the tool's content, construct, and criterion validity (Law, 1987). Therefore, this study focused on initial data collection to determine the E-HAT's test-retest reliability and internal consistency, as well as its concurrent criterion and content validity.

Test-retest reliability refers to the ability of an instrument to produce similar results when administered to the same subjects on separate occasions (Benson & Clark, 1982; Kielhofner, 2006). It is ideal for tests that monitor change over time (Benson & Schell, 1997). Researchers should consider the impact of the first administration on the second (i.e. memorization of answers or improved scores with practice), when deciding if test-retest is an appropriate measurement of reliability for their purposes (Kielhofner, 2006). The Pearson Product Moment (PPM) correlation should be 0.60 or higher for a test to be considered reliable for test-retest (Benson & Clark, 1982; Kielhofner, 2006).

The other method of reliability that is appropriate for the E-HAT is internal consistency. Internal consistency ensures that every item in the instrument is measuring the same construct (Benson & Clark, 1982; Benson & Schell, 1997; Kielhofner, 2006). Cronbach's coefficient alpha (α) indicates the strength of inter-correlation, with a value of 0.80 or higher being most accepted (Benson & Clark, 1982; Kielhofner, 2006). The PPM can also be calculated and is useful in determining the strength of individual items, and should fall in the 0.70-0.90 range (Kielhofner, 2006) to be considered reliable. While reliability is important to assess the accuracy of the E-HAT, this study also focused on two measures of validity to evaluate the interpretation of the assessment.

Content validity examines whether or not the instrument encompasses the adequate depth and breadth of the underlying construct (Carter, Lubinsky, & Domholdt, 2011; Kielhofner, 2006; Nunally, 1978). It is concerned with including relevant material, as well as excluding irrelevant material. The theory behind the construct guides the inclusion or exclusion of specific items (Kielhofner, 2006). Clinicians, patients, and statistical measures can all contribute to establishing content validity (Nunally, 1978).

Criterion validity evaluates how well a new instrument agrees with an instrument that has already established its validity in measuring the same construct(s) as the new tool (Nunally, 1978). Criterion validity can be separated into two types: concurrent and predictive. Concurrent validity simultaneously compares the new instrument with a previously established instrument that measures the same construct. In contrast, predictive validity uses the new instrument in the first round of testing and then uses the previously established instrument to measure change at a later time (Benson & Clark, 1982; Carter, Lubinsky, & Domholdt, 2011; Kielhofner, 2006). For the E-HAT, the researchers chose to calculate concurrent validity in order to compare it to a commonly used assessment: the Beery-Buktenica Developmental Test of Visual-Motor Integration, Sixth Edition (Beery VMI). Higher correlation values in the concurrent assessments will reflect greater validity (Kielhofner, 2006). All forms of validity and reliability are important in instrument development, since their strength determines the efficacy of the instrument in clinical settings.

### Participants

This study utilized convenience sampling of interested volunteers. Participants were recruited through an email invitation sent to the students, faculty, and staff of a small, comprehensive college. The invitation to participate in the study was also extended to family members of students, faculty, and staff, including children (six years and older). The inclusion criteria for the participants were listed as follows: recruited participants had to have their dominant hand ready for functional assessment (i.e. no splints/casts/slings or other restrictions to the upper extremity); participants also needed to have English as their primary language; and participants were required to be six years of age or older to ensure that they had received some formal handwriting education. The invitation elicited fifty-one volunteers to participate in the study. One participant did not meet the inclusion criteria and was excluded. Demographic information for the fifty participants included in this study is presented in Table 1.

TABLE 1
Demographic Information

| | |
|---|---|
| Age | Range 7-75 years |
| | (95% between 19 and 55 years) |
| Gender | 88% Female (N = 44) |
| Hand Dominance | 82% Right-handed (N = 41) |

### Instruments

Electronic-Hand Assessment Tool (E-HAT).

The E-HAT is a software application written in C# programming language for a Windows-based operating system. The E-HAT is intended for use on a tablet computer through a simple software download. The E-HAT program assesses fine motor control with an automated objective assessment measure. It uses copy and trace applications with uppercase, lowercase, and cursive English letters, as well as the Greek alphabet and selected Japanese characters. An objective score is obtained after each character is completed based on speed of writing and pixel-by-pixel accuracy.

The scoring algorithm compares pixels between the target (known as the letter) and the user drawn image (known as the trace). In the scoring summary, the percent of pixel overlap is exactly that—the percent of overlap between the pixels of the letter and the trace. Points are awarded on a graded continuum that allots full points for marks overlapping the target and a lower score for stray marks with progressive decrease in score with increasing distance from the target. Scoring data is stored under a separate file for future reference. For the purposes of this study, researchers used only the assessment portion of the E-HAT tool.

### Beery-Buktenica Developmental Test of Visual-Motor Integration, Sixth Edition (Beery VMI).

The Beery VMI is a standardized paper and pencil assessment designed to assess visual and motor abilities through the copying of a developmental sequence of geometric forms (Beery & Beery, 2006). It has two supplemental tests: Visual Perception and Motor Coordination. These tests are available for people age two and above. The Beery VMI Full Form and Motor Coordination supplement were determined to have the best fit to the E-HAT measures of fine motor control. Researchers practiced administrating and scoring the Beery VMI Full Form and Motor Coordination supplement. Scores were cross-checked with an occupational therapist who has experience in administering and scoring the Beery VMI. Researchers score the Beery VMI independently, using the test manual as a reference. The experienced scorer checks scores intermittently to ensure accuracy.

### Procedures

The researchers gained approval from the Institutional Review Board at Elizabethtown College (Pennsylvania, USA) to conduct the study. Before beginning data collection with participants, the researchers sought to establish initial content validity for the E-HAT. To do this, the researchers systematically categorized all of the font characters available on the E-HAT tool (Arial, cursive, Greek, Japanese) and methodically selected items from each determined category that would be included in each portion of the assessment. The researchers also developed a standardized script to ensure consistent administration of the E-HAT to participants. The study took place in a controlled, standard environment to give each participant a similar testing experience.

After establishing content validity among the researchers, participants were recruited using the procedures outlined in the participants section above. Once participants who met the inclusion criteria volunteered, they were provided with an informed consent form to sign before the assessments were administered. Each participant was assigned a unique identifying number in order to separate his/her name from the data collected. After these preliminary procedures, the E-HAT was administered, followed immediately by the administration of the Beery-VMI. The E-HAT was re-administered at the end of the testing session to record data for immediate test-retest reliability. For participants under the age of eighteen, assent was attained from the child at the beginning of the session and the parent/guardian read and signed the informed consent form. The same testing procedures were followed for pediatric participants as adult participants. A short interview was also conducted with each pediatric participant regarding the E-HAT's ease of use and user impressions to explore the possibility of pediatric use. Each session took 30-45 minutes per participant to complete. Adult participants were invited to return one week after the initial testing to take the E-HAT again in order to establish delayed test-retest reliability. This re-test time took 10-20 minutes per participant to complete.

### Data Analysis

All data was entered into the Statistical Package for the Social Sciences 19 (SPSS), then analyzed using both SPSS and the Statistical Analysis System (SAS). The data analysis included a measure of internal consistency using Cronbach's alpha (α) and measures of concurrent validity and test-retest reliability using Pearson correlations (r). Concurrent criterion validity was measured by analyzing the relationship of test scores between the E-HAT and Beery VMI.

In order to ensure accuracy in the scoring of the Beery VMI, an experienced rater met with the researchers and oversaw practice sessions prior to the initiation of data collection. The rater was also available for questions concerning scoring criteria throughout the research process. Once data collection ceased, the experienced rater scored ten random samples from the fifty participants. Minimal discrepancies were found between the researchers' initial scores and the experienced rater's scores.

### Results

Descriptive statistics were run using the initial E-HAT test results. The mean score on the E-HAT assessment was 55 out of a possible one hundred. The scores ranged from 30 to 71 with 95-percent of participants falling within the scoring range of 38 to 66 points out of one hundred.

The analysis of internal consistency was done using Cronbach's alpha (α) and indicated excellent internal consistency (α=0.91).

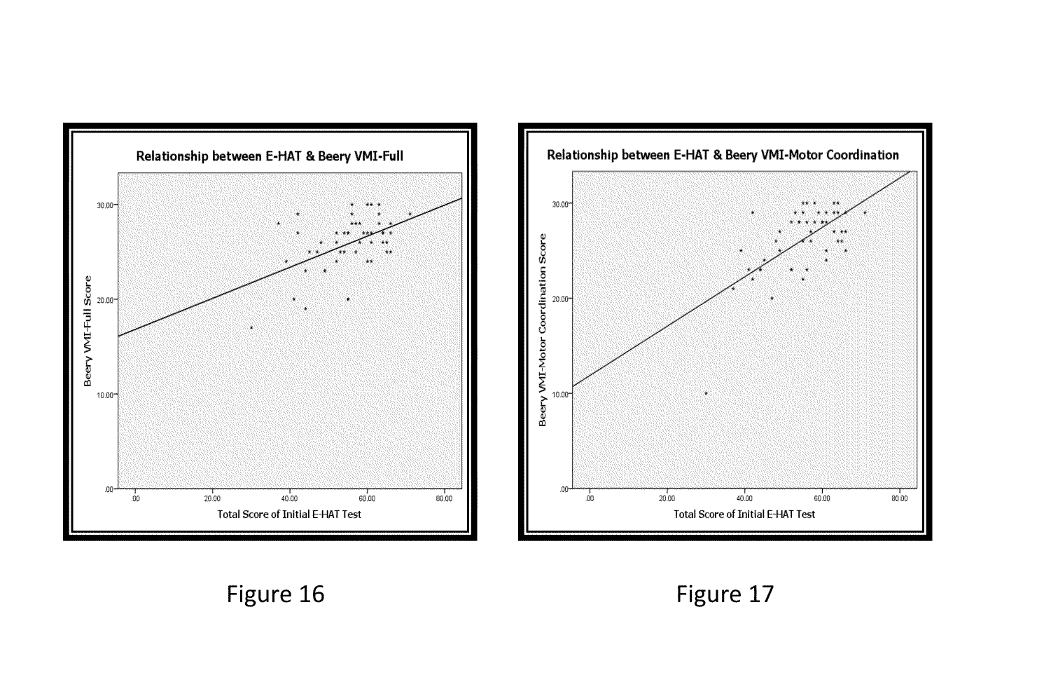

The analysis of concurrent criterion validity was done using a Pearson correlation (r). Concurrent validity was calculated using two measures. The first compared the total score on the E-HAT to the total score on the Beery VMI Full Form (FIG. 16). The E-HAT achieved moderate concurrent validity (r=0.49) when compared to the Beery VMI Full Form. The second measure of concurrent validity compared the total score on the E-HAT to the total score on the Beery VMI Motor Coordination subtest (FIG. 17). The E-HAT demonstrated moderate concurrent validity (r=0.65) when compared to the Beery VMI Motor Coordination subtest.

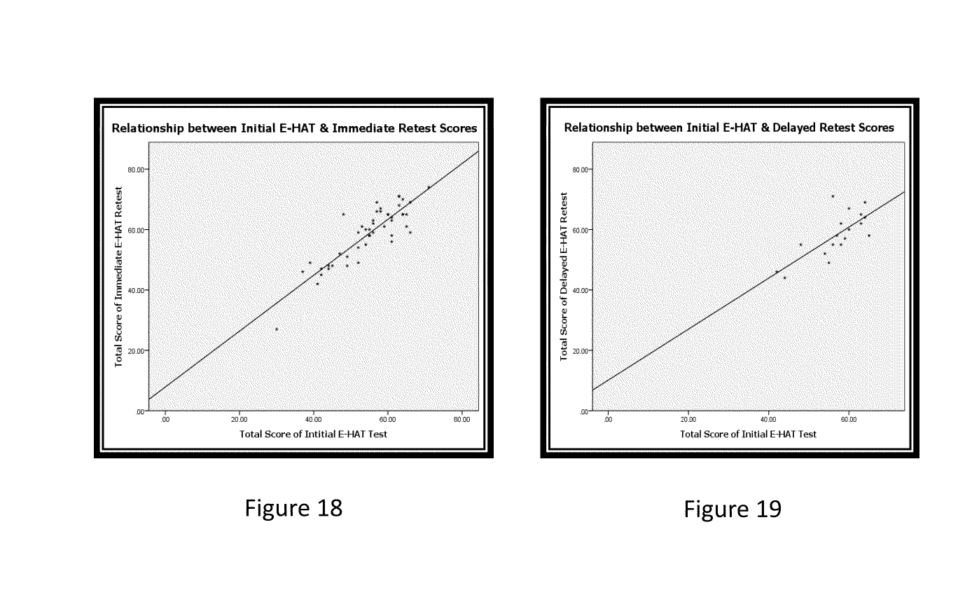

The analysis of test-retest reliability was also completed using a Pearson correlation (r). Test-retest reliability was measured twice, using data from both the immediate and delayed re-administration of the E-HAT assessment. All participants generated immediate retest scores, and eighteen adult participants returned for the delayed retest one week after initial testing. Results show a good correlation between both the initial score and immediate retest and between the initial score and the delayed retest. The correlation between the initial score and the immediate retest score was r=0.88 (FIG. 18). The correlation between the initial score and the delayed retest score was r=0.74 (FIG. 19).

In addition to generating quantitative data, the researchers also collected qualitative data from the four pediatric participants. After completing testing procedures, each pediatric participant was asked a series of open-ended questions: “Is there anything you like about the E-HAT?”, “Is there anything you do not like about the E-HAT?”, and “How would you change the E-HAT to make it better?” Researchers asked these questions because previous studies with the E-HAT have only used adult participants. The current researchers wanted to determine whether the E-HAT would be appropriate for use in a pediatric population or not. Overall, the pediatric users appeared to enjoy the electronic features of the PC tablet and stylus. Participant #202's statement, “I like using the pen as the cursor”, summarizes the general feelings pediatric participants had about the E-HAT. These participants also offered suggestions about how to improve the E-HAT for use with children. Participant #203 commented that, while he did not have trouble completing the tasks on the E-HAT that, “I would change the size and type of letters for younger kids.” Other participants stated that they would change features of the assessment to make it more child-friendly. Participant #201 stated, “I would like it if the computer read the directions to me”, while participant #204 said, “I would like to draw different things [other than the maze].” This initial feedback from pediatric participants will help future development of the E-HAT, particularly to develop a pediatric version of the tool.

### Discussion

This study sought to establish initial reliability and validity statistics for the assessment portion of the E-HAT, a tool that can be used to objectively measure fine motor control through the task of handwriting. The results indicate that the E-HAT has excellent internal consistency, moderate concurrent criterion validity with the Beery VMI Full Form and Motor Coordination subtest, and good test-retest reliability.

The E-HAT's excellent internal consistency rating provides evidence that the tool is consistently measuring the same construct (fine motor control), contributing to its reliability. Because the E-HAT and the Beery VMI are not the same test and do not claim to measure exactly the same construct, a moderate concurrent validity correlation was expected. The E-HAT's good test-retest reliability shows that the test's integrity remains constant over time. The researchers did not expect a large difference between initial and retest scores because the population was typical and had no impairments that affected function; however, the results from this typical population show that the E-HAT has promise as a reliable instrument. These psychometric properties suggest that the E-HAT is an emerging reliable and valid assessment of fine motor control.

The second aim of the study addressed the utility of the E-HAT with the pediatric population. The study included four children (ages 7-12) who gave qualitative feedback about the tool in addition to participating in the other testing procedures. The qualitative interviews suggested that the E-HAT has value within the pediatric population. However, it was determined that the development of a separate, child-friendly version of the E-HAT would be useful to accommodate for the developmental differences between children and adults. The researchers propose that a child-friendly E-HAT could use different computer fonts that are more pictorial in nature (e. g. Wingdings). Because children do not master handwriting until several years after receiving initial instruction (Rosenblum, Parush, & Weiss, 2003), the researchers also propose including even larger characters than are currently on the E-HAT assessment. Additionally, the maze could be changed to a picture that requires a combination of different writing strokes rather than a simple line.

Establishing initial reliability and validity statistics was the main focus of this study; however, some secondary results were also discovered that will contribute to future research using the E-HAT. Because the assessment section was a new addition to the software, a typical scoring baseline had not yet been established. Therefore, it was necessary to pilot the scoring system and generate a typical range of scores that could be used to further refine the E-HAT and provide a baseline for future research. The results indicated that it was very difficult to achieve a perfect score on the E-HAT, with the highest scoring participant receiving 71 out of 100 on the initial test. The study determined that 95% of participants ranged in score between 38 to 66 out of 100 on the E-HAT.

During data analysis, the researchers also discovered that the maze portion of the E-HAT is a unique aspect of the E-HAT software. The maze required participants to have greater fluidity of motion, mobility, and coordination of the entire dominant upper extremity as opposed to the control and mobility of individual fingers or the hand. The Beery VMI Full Form and Motor Coordination subtest had similar task requirements to the other two sections of the E-HAT assessment: copy and trace. The Beery VMI Full Form was similar to the copy section of the E-HAT in which participants needed to copy shapes (Beery) or letters (E-HAT). The Beery VMI Motor Coordination subtest was similar to the trace section of the E-HAT in that participants had to have a certain degree of motor control to either draw within lines (Beery) or trace over characters (E-HAT). No correlating task was found for the maze section of the E-HAT, making it a unique part of the E-HAT tool that requires further analysis.

In addition to the analysis of the assessment portion of the E-HAT, an administration protocol was developed for the E-HAT assessment. In order to standardize administration, the student researchers developed a protocol for both adult and child administration. The protocol outlines set-up and testing procedures that are unique to the E-HAT, including calibrating the computer stylus and running a practice E-HAT session before actual testing occurs. Having the protocol allows consistent administration across raters, and also establishes a baseline for future administrators.

### Implications for Practice

This study is important to occupational therapy because it details the development of an objective fine motor assessment that is easy to administer and consumes minimal additional effort to score and interpret the results. The E-HAT has the potential to be used with a variety of populations (e.g. orthopedics, neurological impairments, pediatrics). Because of its portability, therapists can use it in a variety of settings (e.g. rehabilitation hospitals, outpatient clinics, school-based services), as long as they have access to a tablet computer. The E-HAT has particular use for hand therapists, who routinely address fine motor control with their clients, and school-based therapists who need to evaluate and treat multiple students in a limited amount of time.

### Example #2

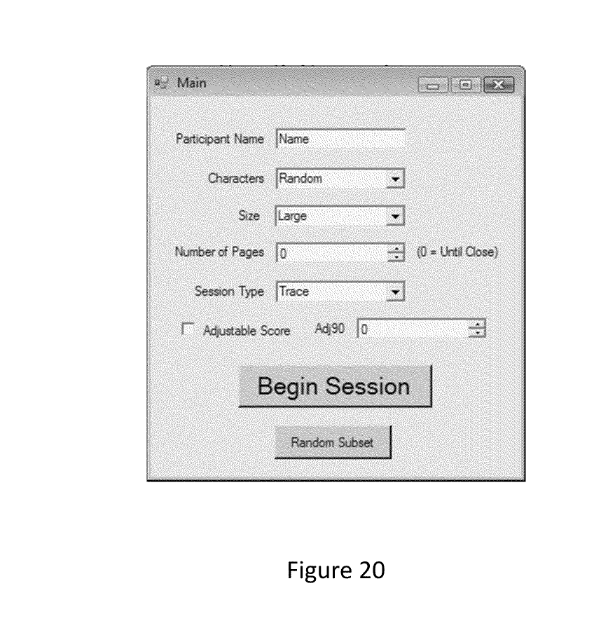

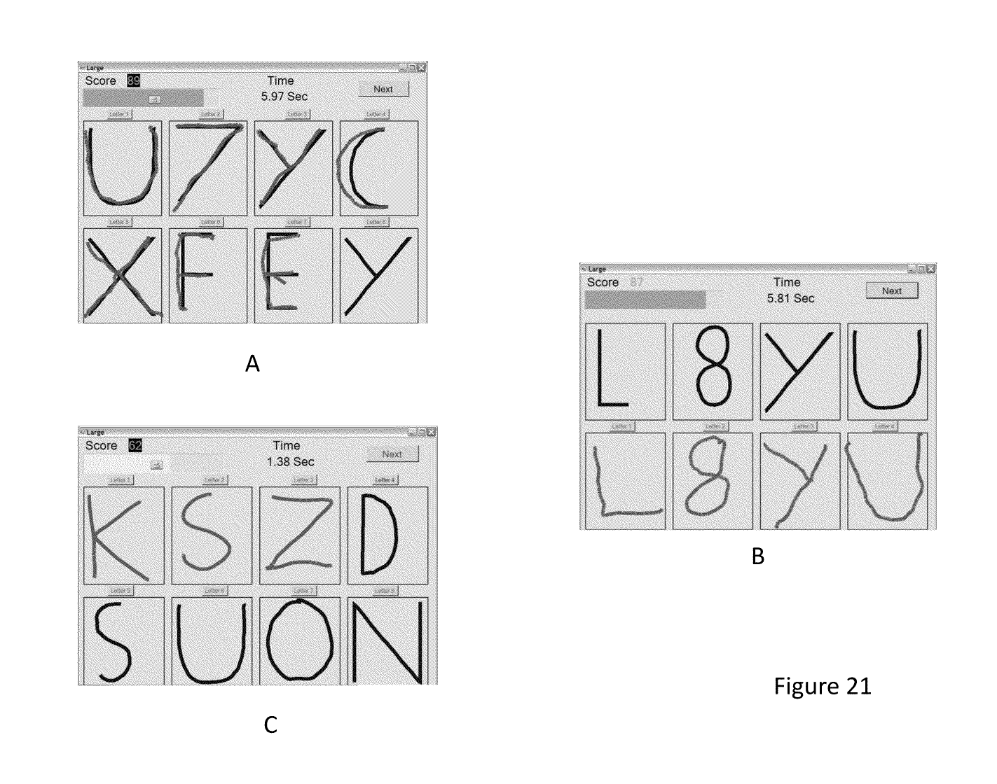

In this example, the E-HAT prototype was written in C# language, which is an evolution of C++. It captures real-time pen position, velocity, and acceleration while subjects perform a variety of fine motor tasks. It has a customizable menu (FIG. 20) with various options for character selection and session types. Three session types, or subtests, are available: Trace, Copy, and Copy/Disappear (FIG. 21). The software can randomly assign letters and numbers for the subtests, or the user can select specific characters via the Characters field or Random Subset feature. The design of the letters and numbers is based on the Handwriting Without Tears® (HWT®) program (Olsen, 2009).

The E-HAT provides two output scores: speed and accuracy. These appear on the screen upon completion of each character. Speed is calculated by subtracting the time the pen starts writing from the time the “Letter” button is selected. Accuracy is computed using an exponential function with input based on the average pixel error of the displayed character and the drawn character. There are three error calculations: display character versus drawn character, drawn character versus displayed character, and general direction/slope/angle versus displayed character. These calculations are averaged and placed into an “Average Error” equation, which yields the overall accuracy score for each character. Upon subtest completion, overall summary scores are reported, including average speed, average accuracy, best accuracy, and worst accuracy.

A first clinical trial involving one adult patient and one certified hand therapist was conducted. The patient responded favorably to the E-HAT, stating it was more motivating to use in comparison to pencil and paper tasks. Likewise, the clinician noted that the tool increased the patient's engagement and interest in the activity. She was concerned, however, that the frequent reporting of speed and accuracy scores could discourage lower functioning patients and negatively impact treatment. She also advised that the height difference between the table-top and the tablet PC surface be eliminated so as to simulate a more natural writing environment.

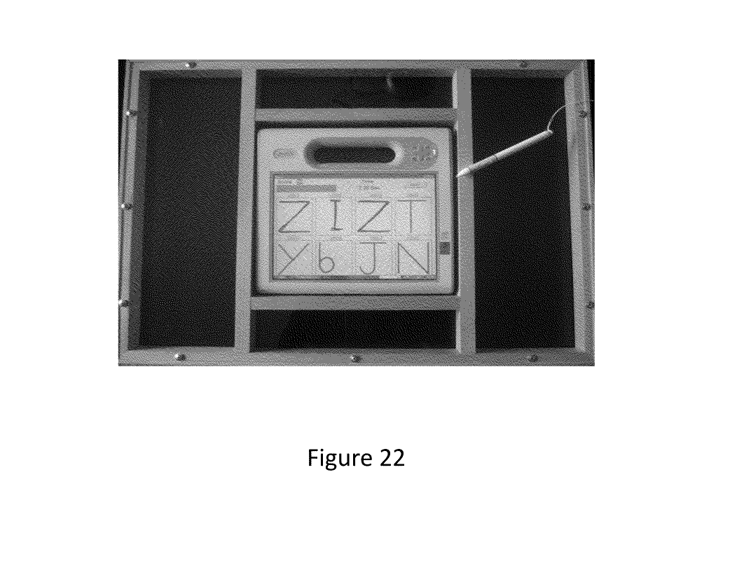

In response, a second E-HAT prototype was designed to include an adjustable score component. When activated this feature conceals patients' actual scores while they are using the tool; clinicians can access the raw data scores after the session is finished. This adjustable score is important as clinicians should use client-centered approaches to increase motivation and facilitate patient success (Gulick, 2009). In addition, a Plexiglas overlay was built to set on top of the tablet PC to create a flat surface when using the E-HAT. The wooden frame is approximately two inches in height (FIG. 22).

In order for the E-HAT to be an effective assessment and intervention tool, clinicians must assess the dynamic interaction between the three components of motor learning theory.

Clinicians must note patients' cognitive, perceptual, and sensorimotor skills; the E-HAT's demands for speed, accuracy, and attention; and the influence of the clinical environment. By assessing all three components, clinicians will be able to select the most appropriate E-HAT subtests, determine if the adjustable score component should be activated, and adapt the environment to support the patients' performances (i.e., their ergonomic factors of body posture, pencil grip, and pencil positioning).

### Purpose of the Study

An adult-aged handwriting rehabilitation tool that provides objective measurements is needed (Faddy, et al., 2008; Gulick, 2009; Rosenblum, Weiss, et al., 2003). Thus, a case study on patient and clinician perspectives of the E-HAT was conducted in order to determine if this software meets the needs described.

### Methods

Descriptive case study methodology was utilized with a mixed methods approach to explore patient and clinician perspectives of the E-HAT. Case studies investigate new areas of inquiry, generate hypotheses for future research, and are strengthened by the collection of both quantitative and qualitative data (Creswell, 2009; Luck, Jackson, & Usher, 2006; McGloin, 2008; Portney & Watkins, 2009; Yin, 1984).

### Participants & Procedures

Participants were selected via convenience sampling methods. First, clinicians at the participating medical institution were contacted, and meetings were arranged based on their availability. During this initial meeting, the case study was introduced and an E-HAT demonstration was provided. Clinicians were asked to recommend patients on their caseloads who met the following inclusion criteria: (a) currently receiving treatment at the facility, (b) impairment of upper extremity due to orthopedic or neurological diagnoses, (c) impairment to dominant hand used for writing, (d) ready for functional assessment as determined by current clinician, (e) age 18 or older, and (f) fluent in English.

The two field researchers met with the clinician-identified patients during their next available therapy appointments. In the initial session, the study was described to the patients. If they volunteered to participate and signed consent forms, the data collection process began. Quantitative data was collected via three assessment tools given to the patients in the following order: Hand Assessment Tool (HAT) (Naidu, Panchik, & Chinchilli, 2009); Beery-Buktenica Developmental Test of Visual-Motor Integration, Fifth Edition (Beery VMI) (Beery & Beery, 2006); and the E-HAT. Qualitative data emerged through direct observations and semi-structured interviews. In follow-up sessions, only the E-HAT was administered in combination with observations and interview questions.

### Instrumentation

HAT.

In addition to collecting demographic and medical history information, this 14-item self-report questionnaire examines patients' perspectives on the impact their wrist and hand injuries have on task performance. Specifically, the HAT investigates patients' perceptions of their activity limitations while executing tasks without using compensatory strategies or adaptive equipment. Answers are based on a Likert scale of one to five, and scores are calculated using a specified equation. Results range from zero to 100, with higher scores indicating greater activity limitations (Naidu, et al., 2009). Attaining patients' perspective is important as patient satisfaction is increasingly becoming known as a measurement of quality of patient care (Chung & Haas, 2009).

Naidu, et al. (2009) published a study supporting the HAT's reliability and validity. Excellent internal consistency was found with Cronbach's alpha of 0.91. The concordance correlation coefficient for test-retest reliability was 0.73, with a 95% confidence interval=(0.60, 0.83). In addition, the Pearson correlation coefficient for validity between the Disabilities of Arm, Shoulder, and Hand Questionnaire and the HAT was high at 0.91, with a 95% confidence interval=(0.88, 0.95). Modest agreement between the HAT and Short Form 12 Health Survey physical score was also noted.

Beery VMI.

The Beery VMI facilitates the identification of visual-motor deficits. It includes three forms, but only the main Developmental Test of Visual-Motor Integration was utilized. The adult version of this 24-item assessment was administered and scored according to guidelines in the test manual. Prior to collecting data, researchers practiced the administration and scoring of the Beery VMI with non-impaired persons in order to increase their feelings of competency when using this tool.

The Beery VMI is widely considered to be the most valid and thoroughly researched test of its kind and can be used with many cultural groups, since individuals copy various geometric forms rather than letters or numbers. The adult version was nationally standardized in 2006 using data from 1,021 adults, aged 19 to 100. Based on a random norming sample of 25 adults, interscorer reliability for the Beery VMI was 0.94; one-week test-retest reliabilities for a sample of 20 persons, ages 60-69, were 0.88. The Beery VMI has extensively documented validity for ages 2 to 19, which appears to be generally applicable to older adults as well. The Beery VMI results for 30 outpatients, ages 55-80, correlated significantly with results from the Wechsler Adult Intelligence Scale-Revised, the Benton Visual Retention Test®, and staff estimates of adaptive functioning (Beery & Beery, 2006).

Researchers administered the Beery VMI because visual-motor integration is an important component of handwriting performance and is strongly correlated with writing legibility (Feder & Majnemer, 2007). Moreover, the Beery VMI served as a comparison tool in order to determine if the E-HAT accurately measured visual-motor deficits.

E-HAT.

The E-HAT was administered according to protocols discussed by the researchers. The first step of the protocol involved an explanation of the tool and demonstration of one page of the trace subtest, set with large, randomly assigned characters. The second step involved entering the unique patient identification number in the “Participant Name” field on the main menu; selecting the same trace subtest with random, large characters; and, choosing two pages. Patients completed the first page of the test without the Plexiglas overlay and the second page with the overlay. The third step of the protocol gave patients the opportunity to practice the trace subtest, complete another E-HAT subtest, or discontinue using the software. If they chose to continue, they had the option of using the Plexiglas overlay.

In follow-up sessions the E-HAT was utilized for intervention rather than assessment purposes; thus, no pre-determined protocols for administration were followed. Subtests were selected based on the motor learning theory and patients' preferences.

Direct Observations.

Researchers observed the patients as they completed the various assessments. Field notes were written based on what was seen. For example, the two field researchers recorded the features of the E-HAT that appeared problematic and well-liked.

Semi-Structured Interviews.

According to Patton (2002), interviewing allows researchers to gather information that cannot be collected through direct observation (e.g., thoughts and feelings). Additionally, asking informal, open-ended questions allows for spontaneity in responses, and having preplanned topics to discuss enhances comparability and reliability of the data (Dereshiwsky, 1999). Thus, researchers utilized an interview guide approach, embedding various questions for patients within each therapy session. A different set of questions were asked to clinicians throughout the study.

### Data Analysis

Quantitative Data.

The researchers coded all data using patient identification numbers to ensure confidentiality. Demographic information and results from the HAT, Beery VMI, and E-HAT were organized in charts using Microsoft® Office Excel® 2007. Patients' scores were descriptively compared to assess the relationship between the E-HAT and the other two assessments. Avoidance of statistical analysis was intentional in order to protect the trustworthiness of the results (Kooistra, Dijkman, Einhorn, & Bhandari, 2009), as well as to maintain focus on the qualitative data and hypothesis generation as is appropriate for case studies (McGloin, 2008). It was believed that if patients exhibited high HAT scores and low Beery VMI scores, they would also have low speed and accuracy E-HAT scores. If no trend between scores was found, then the E-HAT's appropriateness for assessment was questioned.

Qualitative Data.

Researchers wrote field notes during and immediately after sessions. Informal opportunities for member-checking were provided via active listening techniques to strengthen the rigor of the case study (McGloin, 2008). Observations and feedback were analyzed via systematic thematic coding. The two field researchers independently typed their notes and labeled each observation or quote with a specific category name. Related categories were grouped together and an overarching theme label was attributed to each group. Next, the researchers collaborated to discuss and combine the categories and themes. The researchers kept similar themes and generated new themes that fit the combined data; categories were rearranged as necessary to fit into these newly identified themes.

Finally, all data was subjected to peer-review during a research team meeting, which the majority of the authors involved in this study attended. During this time, both quantitative and qualitative results were discussed and made available for critical review in order to ensure the truth value of the data (McGloin, 2008), as well as to allow for further progression of the study.

### Results

Data collection occurred over a two-week period. Five occupational therapy clinicians from a non-profit organization specializing in orthopedic and neurological rehabilitation volunteered to participate; three identified a patient from their caseloads who met the inclusion criteria. All eight participants were females.

Patient 1 was 58-years-old, recovering from a stroke that occurred 96 days prior to her initial session; she presented with right-side hemiparesis. Patient 2 was 48-years-old, suffering from a chronic hand injury. She previously underwent her first of three surgeries on her right hand. Patient 3 was 50-years-old, experiencing chronic inflammation in both upper extremities secondary to diabetes. Her inflammation began prior to the testing. All patients were right-hand dominate. A follow-up session was scheduled with Patient 1; Patients 2 and 3 were only seen once.

### Quantitative Data

Trends indicated that patients' scores on the HAT and Beery VMI were consistent but that scores between these and the E-HAT were inconsistent. Refer to Table 2 for the results from all three assessments.

TABLE 2
Quantitative Scores for Each Patient

| TEST/SCORES | Pt. #1 | Pt. #2 | Pt. #3 |
|---|---|---|---|
| HAT | | | |
| Score | 42.86 | 69.64 | 23.21 |
| Beery VMI | | | |
| Raw | 20 | 19 | 26 |
| Standard | 69 | 50 | 95 |
| Scale | 4 | 1− | 9 |
| Percentile Rank | 2 | 0.07 | 37 |
| E-HAT | | | |
| Trace Subtest | | | |
| Overall Speed ^{a} | 5.92 | 4.41 | 4.01 |
| Overall Accuracy | 83.71 | 96.63 | 96.63 |
| Trace | | | |
| Subtest/Plexiglas | | | |
| Overall Speed | 10.28 | 4.56 | 3.49 |
| Overall Accuracy | 89.57 | 96.38 | 93.25 |
| Copy Subtest ^{b} | | | |
| Overall Speed | 8.77 | 3.67 | 3.38 |
| Overall Accuracy | 55.60 | 69.00 | 85.00 |
| Disappear Subtest ^{b} ^{c} | | | |
| Overall Speed | 6.63 | 3.04 | 3.37 |
| Overall Accuracy | 79.00 | 84.50 | 90.25 |

Note.
^{a} Speed reported in seconds.
^{b} Patient 1 used the Plexiglas overlay for these subtests; Patient 2 & 3 did not.
^{c} Patient 1 completed during second session.

According to their HAT scores, Patient 2 perceived she had the greatest activity limitations and Patient 3, the least. Specifically, Patient 2 cited five tasks as “unable to do”, four as “severely difficult”, three as “moderately difficult”, one as “mildly difficult”, and one as “not difficult”. Patient 1 cited zero tasks as “unable to do”, three as “severely difficult”, six as “moderately difficult”, three as “mildly difficult”, and two as “not difficult”. Finally, Patient 3 cited zero tasks as “unable to do”, zero as “severely difficult”, four as “moderately difficult”, five as “mildly difficult”, and five as “not difficult”.

As would be expected from the HAT results, Patient 2 scored the lowest on the Beery VMI, followed by Patient 1 and then Patient 3. The standard scores of both Patient 1 and 2 on the Beery VMI fell below average, indicating possible visual-motor deficits. Patient 3's standard score was well within the average performance range of 85 to 115.

Finally, E-HAT performances were analyzed. Patient 3 once again performed the best; however, in contrast to trends from the other two assessments, Patient 1 scored the lowest rather than Patient 2. All patients scored the worst in the copy subtests and the best in the trace subtests. Also, regardless of the subtest or patient, accuracy and speed scores were typically indirectly related, such that when patients improved their accuracy, they wrote slower; or when they wrote faster, they became less accurate.

### Qualitative Data

Four main themes were identified by the researchers; these and their associated categories are displayed in Table 3.

TABLE 3
Themes Identified in Patients' and Clinicians' Feedback

| THEMES | | CATEGORIES |
|---|---|---|
| 1. | E-HAT is motivational. | Instant score gratification |
| | | Monitors improvement |
| | | Focus on task rather than pain/therapy |
| | | Game-like and interactive |
| | | E-HAT versus pencil/paper |
| | | Less stressful |
| 2. | Environment impacts | Plexiglas interfered with perception |
| | performance. | Lighting and seating |
| | | Arm and/or wrist support needed |
| | | Stylus versus arrow |
| | | Circumference of stylus |
| | | Force needed to use stylus |
| 3. | Software improvements | Lack of instruction |
| | necessary. | Audio feedback |
| | | Difficult “Letter” button |
| | | Page layout |
| | | Colors and contrast |
| | | Abnormal characters |
| | | Difficulty noting progress |
| 4. | E-HAT market is | Orthopedic versus neurological rehab |
| | expandable. | Patient demographics |

Theme 1: E-HAT is Motivational.

Various observations and quotes indicated the motivational qualities of the E-HAT. First, all participants enjoyed the immediate, objective feedback. Patient 2 stated, “I'm one of those people that like instant gratification. I know how well I'm doing because I can see it on the screen.” This feedback also allowed patients to make personal goals for improving. After attempting the second “L” in a row on her copy subtest, Patient 1 exclaimed, “All right; I increased that one better!” Her facial expressions reflected her performance, such that she smiled when her scores improved and frowned when they did not. She explained, “I like having a target to reach for.”

In addition, the E-HAT allowed patients to shift their focus away from the act of doing therapy or experiencing pain. Patient 3 stated, “The fascination with what I'm doing takes over and I'm less focused on my hand.” Both Patients 2 and 3 likened the E-HAT to a game. “I don't look at it [the E-HAT] so much like a ‘test’,” said Patient 3, “it's more interesting than the other assessment [the Beery VMI]. It keeps my focus better.” When describing the E-HAT, Patient 2 stated, “I actually feel like I'm doing something without thinking about it so much.” Patient 1 shared that she would more likely practice her handwriting using the E-HAT in comparison with pencil and paper, which is similar to the feedback received from the single patient interviewed.

This shift in focus also led to the E-HAT being less stressful. After completing the Beery VMI Patient 2 wiped her palms on her pants; when asked if she needed a break, she responded, “No. That was just a little nerve-racking” In contrast, when completing the E-HAT subtests, the patients typically smiled, laughed, or commented on their scores. Nervousness or stress was not observed.

Theme 2: Environment Impacts Performance.

Notes from direct observations and interviews indicated the impact of several environmental elements on the patients' E-HAT performances, including the Plexiglas overlay, tablet surface, and tablet stylus.

First, the Plexiglas overlay received mixed reviews from the participants. Patient 1 stated that it helped stabilize her arm, but the frame was not wide enough for her to comfortably rest her elbow. Her discomfort was observed as she repositioned the overlay and her body multiple times throughout the session. One clinician suggested the need to restructure the Plexiglas to provide better elbow support. Patient 3 was also observed adjusting her seat height in order to attain better body positioning while using the overlay. She, however, did not like the overlay, stating it “messed up” her perception and interfered with her control. Similarly, Patient 2 complained that the overhead light created a glare making it difficult to see the tablet screen. Clinicians also voiced concern about the glare caused by certain lighting on the overlay.

Second, in regards to the tablet PC surface, Patient 3 noted there was not enough space for her to rest her wrist: “[My wrist] falls right off the tablet when I'm doing the bottom row; the right corner is the hardest.” The tablet screen also interfered with perception. Patient 1 stated, “I am having difficulty knowing where the pen will hit the tablet.” After being advised to look at the arrow on the tablet screen versus the tip of the tablet stylus, her scores improved; she recommended this advice be given to all users of the E-HAT.

Third, regarding the tablet stylus, Patient 1 felt that the circumference of the pen was not adequate: “It is easier to write with a built-up pen. I was taught in OT with a bigger pen.” Both patients and clinicians recognized that the force needed to use the stylus was different than a pen or pencil. Patient 2 stated, “[The stylus] glides smoother and is less painful for me.”

Theme 3: E-HAT Market is Expandable.

Two categories indicated the expandable usage of the E-HAT beyond the current market (i.e., adults with orthopedic and neurologic impairments). All clinicians that treated orthopedic patients stated that handwriting was not typically a focus in their rehabilitation practices. One said, “Handwriting is not usually found to be difficult, and if it is, I tell them [the patients] to practice at home.”

Additionally, participants speculated on the patient demographics that would be most suited for the E-HAT. Patient 3 indicated that her teenage son, who was on the Autism spectrum, would especially benefit from this tool. One clinician was interested to see if her teenage patient would like the E-HAT despite his unfamiliarity with technology due to his rural location. Another clinician felt the E-HAT would be well-liked among the pediatric population, since this younger generation was very adept with the use of technology.

### Discussion

Analyses of technology's barriers and benefits in health care have primarily relied on the clinician's perspective (Andre, et al., 2008; Brown & Coney, 1994; Crounse, 2009; Hewlett-Packard, n.d.; Motion Computing, 2006; Shortliffe, 2005). While clinicians' attitudes are important, it is also imperative to consider the patients' viewpoint on technology. This case study enabled the consideration of both perspectives. Analysis of the data yielded various strengths of the E-HAT, several weaknesses of and recommendations for the E-HAT, and information on the E-HAT's potential for rehabilitation.

### Strengths of the E-HAT

Motor learning theory stresses that learning requires both intrinsic (e.g., visual) and extrinsic (e.g., verbal) feedback. It also suggests effective therapy involve timing individuals as they repeatedly practice functional tasks (Shumway-Cook & Woollacott, 2001). The E-HAT provides this feedback, timing, and repetition.

As noted in Theme 1, all patients felt the E-HAT's immediate scores were motivating since these provided means for setting personal goals for improvement. The various character sizes and subtests available also provided means for challenging their abilities. In addition, patients found it easier to stay on task while using the E-HAT. When completing the Beery VMI, the patients were unsure how they would be graded or what they would be graded on, which appeared to increase their nervousness. Thoughts that prevent someone from focusing on and successfully completing tests include predictions of failure, self-degrading thoughts, or preoccupation with the consequence of doing poorly (Understanding Academic Anxiety, n.d.). In contrast, when completing the subtests on the E-HAT, the patients knew that they were being tested on speed and accuracy; thus, they focused better and experienced less test anxiety.

Lastly, today's health care world demands efficiency and accuracy of documentation (Andre, et al., 2008). The E-HAT, with its immediate, objective scoring, provides a means for both. The weaknesses of subjective assessment tools were perceived while scoring the Beery VMI. This test not only required additional time to score after treatment sessions but familiarization and experience with the scoring procedures to ensure tests were scored correctly. The time demands and relativity of subjective tools were avoided when using the E-HAT, further supporting the literature which states that objective analyses are superior to subjective (Coley, et al., 2008; Menegoni, et al., 2009; Raiss, et al., 2007; Rosenblum, Weiss, et al., 2003).

In summary, this case study indicates the E-HAT has various strengths. These include providing feedback through speed and accuracy scores, increasing patient motivation to do therapy, offering client-centered options through customizable menus, reducing test anxiety, and lessening clinician workload and improving documentation via objective scoring.

### Potential for Rehabilitation

Kotani and Horii (2003) found that tablet PCs are easy to learn to use and more efficient than traditional mouse-controlled computer technology. Beneficial factors of technology for both the clinician and the consumer in health care units include improved productivity and quality of care (Andre, et al., 2008.; Crounse, 2009), reduced errors (Andre, et al.; Crounse), increased patient education and communication in the clinic (Crounse; Motion Computing, 2006), and improved clinical data collection (Motion Computing). Therefore, the future acceptance of an innovative, electronic tool like the E-HAT in occupational therapy clinics looks promising.

The results from this case study indicate that the E-HAT is an effective assessment tool for adult handwriting. Unlike the Beery VMI raw scores indicate, the field researchers felt that Patient 1's fine motor and visual-motor skills were more impaired than Patient 2's. Out of the 24 shapes copied on the Beery VMI, Patient 2 made 17 forms correctly, while Patient 1 only made 15 correctly. Additionally, when the patients completed the Beery VMI, there were noticeable differences in their handwriting skills. Patient 2 copied everything with straight, bold pencil strokes, while Patient 1 wrote slower and more unsteadily, creating wavy pencil lines. Therefore, since Patient 1 scored lower on the E-HAT tests, the researchers feel that the E-HAT did an accurate job of assessing handwriting skills. Nevertheless, no comment can be made on the effectiveness of the E-HAT to improve handwriting skills. Future long-term studies that monitor patient use of the tool must be conducted in order to make that determination.

Due to the feedback related to Theme 3, the population to which the E-HAT would be most appropriate may need to be expanded. The E-HAT was originally intended for patients with orthopedic and neurologic impairments. The researchers found that handwriting is not typically a priority during rehabilitation with orthopedic patients but that it is with neurologic patients. Literature supports this finding in that handwriting has been shown to be problematic for adults with strokes and brain injuries (Faddy, et al., 2008). Thus, the E-HAT's clinical applicability will need to be adjusted since the frequency of E-HAT use will most likely be higher among patients with neurologic impairments.

The E-HAT was also geared toward the adult population. Although strokes are typically considered a disease of the elderly, they are a potential risk for everyone, including children. The side-effects of strokes in children are generally the same as in adults, including hemiparesis, hemiplegia, unilateral neglect, visual-field cuts, decreased visual perception, and decreased cognition (American Heart Association, 2010). Therefore, the age applicability of the E-HAT will need to be expanded.

Furthermore, feedback from participants indicated that the software may appeal more to the generations who grew up with technology and that it may be difficult for the elderly to use. As a result, the most likely users of the E-HAT may be the younger generations and the pediatric population. Nevertheless, the baby boomer generation is a large portion of healthcare consumers and has been found to be more adaptable, technologically perceptive, and open to innovation in comparison to previous generations (Wister, 2009). Thus, using the E-HAT with the current middle-aged adult population, who will make up the future generation of elderly, still seems appropriate.

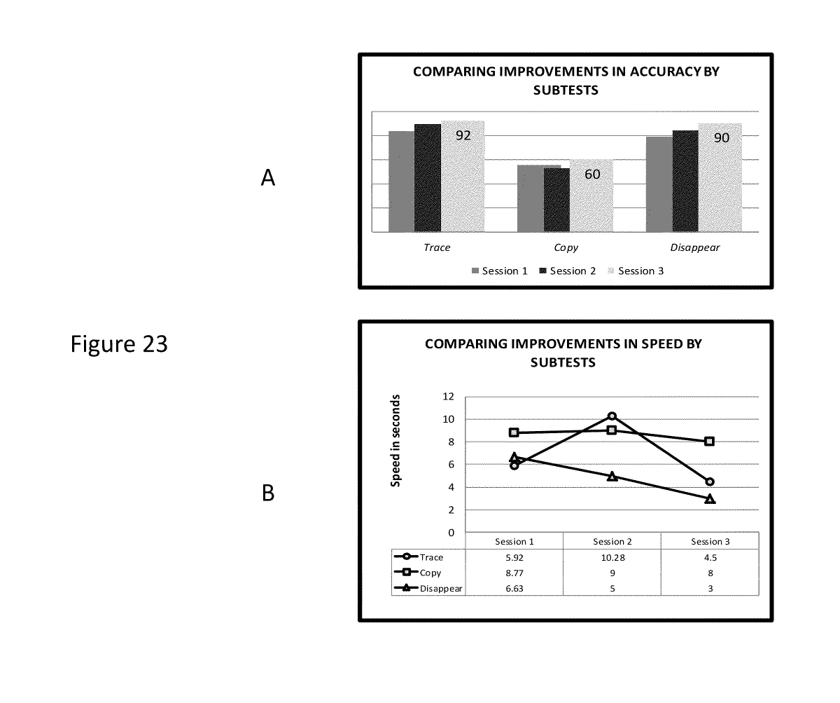

In summary, the potential for using the E-HAT in rehabilitation depends on the patient's diagnosis, age, and familiarity with technology, but the E-HAT's applicability across generations is highly possible. As a result, this study supports the need to expand the E-HAT's targeted market population. Graphical representations of E-HAT Summary Scores are depicted in FIG. 23.

### Example #3

The E-HAT software (also referred to herein as E-HAT I) was edited through the development of the following features to enhance the utility of the assessment and intervention tool (referred to herein as E-HAT II).

### Scoring

The E-HAT II software is equipped with an overall different coding system. While the E-HAT I was composed of repetitive codes, the new software was re-written to allow for the user interface to be more simplistic and user-friendly. The scoring algorithm was re-designed to enable more accurate scoring and is currently comprised of a pixel overlap ratio. This examines the amount of the original letter that is overlapped, how much is written outside of the lines of the original letter, and the percentage of how much of the original letter is colored in. With these new scoring codes, the patient's handwriting abilities may be more accurately assessed. The recommendations made also enabled the software to save overall scores for each subtest session within an individual patient folder, allowing for the tracking of patient progress.

### Menu Options and Formatting

The first noticeable change between the E-HAT prototype and the E-HAT II prototype is the main menu. This updated version of the E-HAT allows the patient to have more choices, thus allowing the therapist to create a session that is most appropriate for and meets the needs of his/her patient. The main menu continues to permit adjustable scoring for patient presentation, as well as the ability to choose the size of the characters, with the range includes 5 different sizes of small, medium-small, medium, medium-large, and large. The previous characters utilized were those adapted from the Handwriting Without Tears® program, a developmental handwriting program for elementary aged children (Olsen, 2009). To cater to clientele and to ensure continued patient motivation, four different character fonts and numbers were added: Ariel, Greek, Katakana, and Cursive. The Greek and Katakana are primarily comprised of mostly symbols. Since traditional letters are imprinted in memory, indicating implicit motor learning, the inclusion of the symbols allows for the patient to incorporate their visual motor coordination skills to complete the character and facilitate explicit learning (Boyd & Winstein, 2006; Lesensky & Kaplan, 2000; Shumway-Cook & Woollacott, 2001). In addition, there is now an area in which to select the type of set of characters one wishes to use during the session. The variety of sets includes: all letters, upper-case only, lower-case only, and numbers only.

The general format of the activity page/subtest page was re-designed to allow for cueing and more space to complete the intended activity. As recommended, the speed and accuracy scoring of each individual character was shifted from the top of the page to the bottom to shift the character boxes up and create more surface area for the client to rest their wrist while completing each character. The ‘letter’ buttons at the top of each character, which were utilized to prompt the patient to begin, were also replaced with the new titles of ‘Start’ and ‘Stop’. The initiative to include a color scheme, green for the ‘Start’ and ‘Next’ buttons and red for ‘Stop’, was taken to provide visual cues for reminding the client to complete this necessary task before moving on to the next character. Auditory cues were also incorporated at this time, providing the patient with a tone if the patient attempts to proceed to the following character before selecting the red ‘Stop’ button.

### Subtests

Despite the removal of the Copy/Disappear subtest, subtle changes were made to enhance the utility of all the established tests. The color contrast for the characters was altered to black and blue to allow for a more easily visible pixel contrast between the original character and the patient drawing. Also, in accordance for the need of reference points for the ‘Copy’ subtest, 4×4 grids were placed within the area designated for the copied character. To supplement for the Copy/Disappear subtest that was removed, a new subtest element was added entitled ‘Maze’. The maze subtest, which includes six mazes of random difficulty levels, was created to encompass the functions of visual motor coordination, which is also imperative to the activity of handwriting.

Beyond the re-design of the software was the need to replace the plexiglass. An ‘L’ shaped structure was created as the plexiglass replacements and can be placed on either the left or right of the tablet PC depending on hand dominance. It has no plexiglass, which eliminates a glare on the tablet PC screen and also allows for the necessary wrist support that is needed while engaged in any E-HAT II activity (Panchik, Ensminger, & Franklin, 2010; Shumway-Cook & Woollacott, 2001).

### Handwriting and Motor Control

Due to the fact that movement develops through an exchange of various systems, a rehab intervention tool must be dynamic in nature and responsive to change within various environments and contexts (O'Brien & Lewin, 2008; Smeulders, Kreulen, & Bos, 2001). The overarching control that directs this dynamic exchange of movement is known as motor control; “ability to regulate or direct the mechanisms essential to movement” (Shumway-Cook & Woollacott, 2007, p. 4). Since handwriting incorporates highly controlled and individualized movements in order to produce letters (Sallagoity, Athenes, Zanone & Albaret, 2004), the (re)learning process of handwriting is most effectively facilitated by the following components.

### The Natural Contexts

Movements performed in their natural context(s) are more likely to facilitate the achievement and or modification of the intended movement, by causing the individual to spontaneously adapt and problem solve, which reinforces motor control (O'Brien & Lewin, 2008). Thus leading to the ultimate goal of motor control intervention: to increase effectiveness in a variety of environments and context (O'Brien & Lewin, 2008; Sallagoity, Athenes, Zanone & Albaret, 2004; Shumway-Cook & Woollacott, 2007). With this being known, a handwriting intervention tool and assessment should begin by addressing the functional components of handwriting, in order to allow for transferability to the individual's contexts and or environments, whether it may be work, home, school etc. (Feder & Majnemer, 2007; Gulick, 2009; Tam, Ryan, Rigby, & Sophianopoulous, 2009).

### The Occupation

Performance of the occupation itself is preferred in comparison to its component parts (O'Brien & Lewin, 2008). Thus reinforcing that motor tasks are best learned and transferred to functional activities when they are taught within the context of the activity in its entirety (O'Brien & Lewin, 2008; Shumway-Cook & Woollacott, 2007). Handwriting (re) training, for instance, is most appropriately addressed through a task-oriented approach, such as practicing letter formation to prepare for functional writing tasks: writing lists, letters etc., in comparison to individual letter strokes. Practicing letter formation should progress through a sequence of less-to more challenging letter formations, which strengthen motor skills by way of kinesthetic and visuomotor processes (Feder & Majnemer, 2007; Gulick, 2008; O'Brien &Lewin, 2008; Shumway-Cook & Woollacott, 2007; Smeulders, Kreulen, & Bos, 2001). The practicing of the occupation, handwriting, itself can potentially lead to an increase in legibility and or accuracy, but can also decrease with speed, as speed and accuracy are the two most critical components of handwriting that influence overall occupational performance (Faddy, McCluskey, & Lannin, 2008; Feder & Majnemer, 2007; Smeulders, Kreulen, & Bos, 2001).

### Meaning

Motor control is learned best and managed when the task is meaningful. Meaning and drive to perform a task can increase motivation and ultimately improve performance (O'Brien & Lewin, 2008). As handwriting is a major occupational task, it is important to create rehab interventions and assessments that are age appropriate and meaningful to the adult and that encourage participation and increase self-confidence. Many adults feel self-conscious about (re)learning handwriting, due to the perception that handwriting has a strong correlation to intelligence (Feder & Majnemer, 2007; Gulick, 2009; Weisser-Pike, 2005). With this being known, it is imperative to structure interventions that increase motivation and promote a favorable outcome (Gulick, 2009).

In summation, the dynamic interaction between the natural context, occupation, and meaning are essential in creating a holistic handwriting intervention in the natural context that is meaningful to the client (O'Brien & Lewin, 2008; Shumway-Cook & Woollacott, 2001). Clinicians should consider all three components when structuring an effective handwriting intervention with the components of motor control (Feder & Majnemer, 2007; Gulick, 2009; O'Brien & Lewin, 2008; Shumway-Woollacott, 2001).

### Purpose of the Study

The continued need for an adult age-appropriate handwriting intervention tool is imperative, as literature supports the apparent lack of such tool available in practice (Faddy, McClusky, & Lannin, 2008; Gulick, 2009). With this noted, a continued case study on patient and clinician perspectives of changes made to the E-HAT II prototype software in order to assess its clinical applicability is provided. The purpose of this research study was to gather patient and clinician perspectives of the E-HAT II prototype in order to re-determine its applicability to adult handwriting rehabilitation, to determine its applicability as motor control assessment and intervention tool and to further develop the software to make it more clinically useful.

### Methods

Descriptive case study methodology with a mixed methods approached was used to explore patient and clinician perspectives of the E-HAT II prototype software. A case study approach was utilized in order to investigate new areas of study, generate hypotheses and is an effective approach for collecting both quantitative and qualitative data (Creswell, 2009; DePoy & Gitlin, 2005; Luck, Jackson, & Usher, 2006; Portney & Watkins, 2009). Institutional Review Board (IRB) approval was obtained from small comprehensive college and the participating medical institution, in order to conduct this study.

### Participants & Procedures

Participants were selected through convenience sampling with the targeted number of participants as two, both of which were of the neurological impairment population. To begin the research process, clinicians from the medical institution were contacted and meetings were arranged to introduce the E-HAT II prototype to the facility and the therapists. A demonstration of the E-HAT II and a description of our case study were also provided during our initial meeting with the therapists. Clinicians were asked to recommend patients that met the following pre-determined inclusion criteria: (a) currently receiving treatment at the facility, (b) impairment of the upper extremity due to orthopedic and/or neurological diagnoses, (c) impairment of the dominant hand used for writing, (d) ready for functional assessment as determined by current clinician, (e) age 18 or older, and (f) fluent in English (Panchik, Ensminger, Ford, Patrick & DeGoede, 2010).

The researchers met with the recommended participants when available, possibly the following treatment session post-contact. During this time, a description of the study was described to the patient and if he/she were willing to participate, consent forms were signed/collected and the data collection process began. Quantitative data was then collected via the following three assessments and in this order: Hand Assessment Tool (HAT) (Naidu, Panchik, & Chinchilli, 2009); Beery-Buktenica Developmental Test of Visual Motor Integration, Sixth Edition (Beery VMI) (Beery & Beery, 2010) and its subtests of Visual Perception and Motor Coordination (Beery & Beery, 2010); and the E-HAT II. Qualitative data was then collected through direct observation and semi-structured interviews (Panchik, Ensminger, Ford, Patrick & DeGoede, 2010).

### Instrumentation

The Hand Assessment Tool (HAT). This 14-item self-report questionnaire is classified as a measurement of activity limitation. It examines the patient perspectives on the impact of their wrist and hand impairments, specifically their perceptions of activity limitation while completing designated tasks without compensatory techniques or adaptive equipment. Questions are based on a broad spectrum of health related ‘outcomes’, including self-care, leisure, symptoms, and aesthetics. Scoring is based on a Likert scale of one to five, one indicating no difficulty in completing a task and five indicating a complete inability to complete the task. Scores are calculated utilizing a specific equation and range numerically from zero to 100, with the higher score indicating greater limitations during activity. However, if more than two items are missing then the test should not be scored (Naidu, Panchik & Chinchilli, 2009).

A study of the reliability and validity of the HAT was published by Naidu, et al. (2009), exhibiting excellent internal consistency with Cronbach's alpha of 0.91. Test-retest reliability yielded a concordance correlation coefficient of 0.73, with a 95% confidence interval=(0.60, 0.83). Also, the Pearson correlation coefficient between the HAT and the Disabilities of Arm, Shoulder, and Hand Questionnaire (DASH) was high at 0.91, with a 95% confidence interval=(0.88, 0.95).

Beery VMI & supplemental tests: Visual perception and motor coordination. The Beery VMI main test, Development Test of Visual-Motor Integration, was designed to assess the extent to which individuals can integrate their visual and motor skills and facilitates the identification of visual motor deficits. The supplemental tests of Visual Perception and Motor Coordination were created as separate evaluations of visual and motor coordination components. They aim to further investigate the results of the Beery VMI, as it is recognized that those who perform poorly on this assessment may not be able to integrate the visual perception and motor coordination domains, but function adequately when they are separate. The test as a whole is culturally free as it can be used within all ages and cultural backgrounds. It recognizes that those from different backgrounds have varying degrees of experience with letters and numbers, so alternatively, geometric shapes are used. All three forms utilize the same geometric shapes with varying sizes for material conservation (Beery & Beery, 2010).

All three tests were standardized on the same national sample of 2,512 individuals and although the validity of the Beery VMI is extensively documented for those aged 2-19, it was found to be generally applicable to older adults (Beery & Beery, 2010). To maintain valid results, the Beery VMI must be given in the order in which it was standardized; beginning with the main Developmental Test of Visual-Motor Integration, followed by the Visual Perception subtest and ending with the Motor Coordination subtest (Beery & Beery, 2010).

### Direct Observations

Researchers observed the patients as they complete their multiple assessments. Field notes were recorded based upon observations, for example recording areas of the E-HAT II that the patient's found difficult or the areas that they found beneficial.

### Semi-Structured Interviews

Semi-structured interviews allowed researchers to gather information that could not obtained via direct observations, such as a participant's thoughts and feelings (Patton. 2002). Also, asking informal, open-ended questions promoted natural responses from participants and enhanced the reliability and comparability of the data collected (Dereshiwsky, 1999). The interview guide that was used to collect such data was developed by the previous set of researchers and was used in this study, in order to maintain consistency within the collection of the qualitative data (Ensminger, Ford, Patrick & DeGoede, 2010).

### Data Analysis

Quantitative data. The researchers ensured patient confidentiality by coding all data through patient identification numbers. Microsoft Office Excel 2007 was used to construct charts of all testing data collected. This allowed for accurate comparisons of the relationships between the HAT, Beery VMI & Subtest, and E-HAT II scores. Statistical analysis of this data was avoided to protect the reliability of the results and to maintain the integrity of the case study methodology.

Qualitative data. Field notes were written by the researchers during and immediately following the sessions. The data collected through observations and clinician/participant feedback was then categorized by systematic thematic coding. Each researcher coded results of the qualitative data collection process individually and then collaborated their findings to ensure consistency throughout. Together, the researchers created categories that applied to the newly established themes.

### Results

Data collection occurred over a one-week time period. Two occupational therapy clinicians volunteered to participate in this study and each clinician identified a candidate with a neurological diagnosis who met the inclusion criteria. Both participants were seen for one session to complete testing with the HAT and Beery VMI and subtests, which required forty minutes, and to complete the E-HAT II, which required twenty minutes, as well as participate in semi-structured interviews.

### Participant Information

At the time of the study, participant 1 was a 58-year-old male who suffered from a cerebral vascular accident or stroke in 2007. He presented with right hemiparesis and was right hand dominant prior to the stroke. However, he had learned compensatory techniques using his left hand for most daily tasks but completed testing using his affected right hand. Participant 2 was a 40-year-old female who suffered an anoxic stroke in 2001. She was also right hand dominate prior to and after the stroke and presented with both physical and cognitive deficits. Cognitive deficits noted were observations of impulsivity, impaired short term memory, and difficulty following directions during the session.

### Quantitative Data

Trends indicated that participants' scores on the HAT were inconsistent with the other tests. For example, while participant 1 indicated no problem with handwriting skills in the HAT, participant 1 demonstrated challenges with the Beery VMI and accuracy of the E-HAT II subtests. Trends also suggested that the Beery VMI, supplemental tests, and E-HAT II were consistent, indicating some level of impairment with handwriting skills. Refer to Table 4 and Table 5 for the results from the assessments.

TABLE 4
Quantitative Scores of the HAT and Beery
VMI and Subtests for Each Participant

| Test/Scores | Pt. #1 | Pt. #2 |
|---|---|---|
| HAT | | |
| (Low Score = Better Performance) | | |
| Score | 17.308 | 11.61 |
| Beery VMI: | | |
| (Higher Score = Better Visual-Motor Skills) | | |
| Raw Score | 17 | 20 |
| Standard Score | 45 | 58 |
| Scale Score | 1− | 2 |
| Percentile Rank | .02 | .6 |
| Beery VMI: Visual Perception | | |
| (Higher Score = Better Visual Perceptual Skills) | | |
| Raw Score | 26 | 28 |
| Standard Score | 95 | 97 |
| Scale Score | 9 | 9 |
| Percentage Rank | 37 | 42 |
| Beery VMI: Motor Coordination | | |
| (Higher Score = Better Motor Skills) | | |
| Raw Score | 16 | 16 |
| Standard Score | — | — |
| Scale Score | — | — |
| Percentage Rank | — | — |

TABLE 5
Quantitative Scores of the E-HAT II for Each Participant

| Test/Scores | Pt. #1 | Pt. #2 |
|---|---|---|
| E-HAT II | | |
| Trace Subtest | | |
| Arial | | |
| Overall Accuracy | 67 | 73 |
| Overall Speed | 9.73 | 13.05 |
| Katakana | | |
| Overall Accuracy | 70 | 70 |
| Overall Speed | 9.38 | 22.82 |
| Copy Subtest | | |
| Overall Accuracy | 36 | 50 |
| Overall Speed | 11.76 | 22.60 |
| Maze Subtest | | |
| Overall Accuracy | 62 | 88 |
| Overall Speed | 65.39 | 146.28 |

HAT scores. When scoring the HAT, it is important to remember that the lower the score, the better perceived hand and wrist function by the patient. Scoring is based on a Likert scale of one to five, one indicating no difficulty in completing the task and five indicating a complete inability to complete the task. Participant 1 scored a 17.308% perceived activity limitation in overall hand functioning; however, while selecting the question most directly pertaining to this study, “Have you had difficulty writing”, participant 1 indicated “no difficulty”, which has a numerical value of one. In contrast, participant 2 scored a 11.61% perceived activity limitation overall and selected the question most directly pertaining to this study, “Have you had difficulty writing” as “mild difficulty”, which has a numerical value of two. According to their HAT scores, participant 1's score indicated greater perceived overall activity limitation compared to the scores from participant 2, but greater perceived ability in handwriting skills.

Beery VMI and subtest scores. Participant 2 scored higher than participant 1 on both the Beery VMI and the Visual Perception supplemental test, but both raw and standard scores for the Beery VMI were well below the average range of 90 to 109 for both participants, thus indicating potential visual motor deficits. The scores for both participants on the visual perception subtests were high, with participant 2 scoring a 28 out of 30, only two points higher than participant 1. Both participants produced a raw score of 16 on the Motor Coordination supplemental test, which did not yield a standard, scaled or percentage rank, due to the 5 minute time constraint. It has been noted by Beery & Beery (2010) that adults tend to do less well on the timed motor coordination subtest because they work more slowly, which is attributed to why the standard, scaled and percentage ranks were not given.

E-HAT II scores. Finally, the E-HAT II scores were interpreted based on previous research and data analysis using this tool. It was hypothesized by researchers in previous studies that participants who completed the E-HAT II tests faster would produce lower accuracy scores than those who took their time (Panchik, Ensminger, Ford, Patrick, & DeGoede, 2010). Similar to the scores received on the other assessments, participant 1 had the most difficulty with these tests. While he completed the tests at a much faster pace than participant 2, his accuracy scores were lower overall. These results further confirm with the previous study that speed and accuracy are indirectly related; participant 2 receiving higher accuracy scores but slower times than participant 1. Both participants, however, had the most challenges with the ‘Copy’ subtests in terms of speed and accuracy, and the best scores on the ‘Trace’ subtest. The ‘Maze’ subtest produced scores that may indicate an accurate measure of motor control, as both participants' scores were relative to those received on other E-HAT II tests.

### Qualitative Data

Thematic coding was used to analyze qualitative data collected. The purpose of qualitative data collection for this study was to gather participant and clinician feedback on the E-HAT II software and allow for further revisions to enhance the E-HAT II's clinical utility. Themes were produced via field notes that were collected during and after the sessions and through patient and clinician interview questions. Common trends were noted among interviews and field notes, which assisted in the production in the following four overarching themes and their sub-categories, as illustrated in Table 6.

TABLE 6
Themes Identified Through Participant and Clinician Feedback

| Themes | | Sub-categories |
|---|---|---|
| 1. | The E-HAT II is | Instant feedback |
| | motivational | Monitors improvement |
| | | Increase confidence |
| | | E-HAT versus pen/paper |
| 2. | The environment impacts | Seating and table height |
| | performance. | Circumference of stylus |
| | | Sensitivity of stylus |
| | | “L” support restricted ROM |
| 3. | Software improvements | Size of start/stop button |
| | are necessary. | Color contrast for the colorblind |
| | | Start/stop button sensitivity |
| | | Lack of instruction |
| 4. | Normative data as the | Reference for score comparison to |
| | next step of research. | normal population |
| | | Increase ease of understanding scores |

Through various observations and statements made by the participants, it was indicated that the E-HAT II software continues to be motivational. Participants reported increased confidence in their handwriting ability through the visualization of their written character over the computerized character. Participant 1 stated, “I didn't think I would do as well as I did, but the blue made it easy to see how accurately I was tracing the letter.” Also, immediate feedback continued to motivate the participants to continue the use of the E-HAT II in order to better their scores. Participant 2 said, “I like that I can immediately see how I did and try to better my scores the next time. I feel like this test is focused more on the areas that I am weak in.”

In addition, both the participants and the clinicians described the E-HAT II's motivational qualities in that it is more time efficient in comparison to pen and paper assessments for handwriting. One clinician declared, “The E-HAT II is able to assess a patient's performance efficiently, which is much more useful for therapists in a rehabilitation setting. It is also easy for the patients to use, and the idea of a tablet PC is more exciting than a dated pen and paper assessment.”

### Discussion

This case study incorporated both patient performance and patient and clinician perspectives of the E-HAT II, through direct observation and semi-structured interviews. The analysis of this information yielded areas that highlight the E-HAT II's numerous strengths and various weaknesses and provides recommendations for future changes and uses of the E-HAT II, and ultimately considerations for the potential viability of the E-HAT II software for rehabilitation.

### Strengths of the E-HAT II

Various strengths were acknowledged by both participants and clinicians. The following strengths highlight the areas in which the E-HAT II meets consumer and professional demands.

The first strength is that the E-HAT II provides instant feedback and motivation. Both participants felt that the immediate availability of scoring was motivational because it allowed them to visualize their weaknesses and instantly identify areas for improvement. Participants were able to monitor their performance and worked to improve scores based upon immediately received scores. Participant 1 indicated that the instant feedback increased his confidence in his handwriting ability and motivation to continue using the software.

The next strength identified was the improved objectivity of scoring by the E-HAT II, with some potential implications for improved score accuracy. Unlike the previous software, the new scoring mechanism is comprised of a pixel overlap ratio. This investigates the amount of original character overlap, how much is written outside the lines of the original character, and the percentage of how much the original character is colored. Through comparison of patient scores from both studies, it was identified that patients in the previous study received overall higher scores for speed and accuracy with a less restrictive scoring criteria (Panchik, Ensminger, Ford, Patrick, & DeGoede, 2010). Pixel overlap ratio was not programmed in the first software prototype, meaning that participants would to be awarded scoring points for copying the character, even if it was not directly representative of the original character. Now, the scores may consequently be more indicative of true handwriting and motor control accuracy and ability. In conjunction with the scoring changes, the alteration of character letters from the Handwriting Without Tears program was also changed, and may have also assisted in more accurate scoring, as the characters are more indicative of adult handwriting.

The third strength is the storage mechanism which enables patient scores to be housed in individual patient folders. The storage of data allows for the patient's scores to be housed in one place for comparison and progress monitoring, which the study has proven to be useful in research and in rehabilitation in the future.

The forth strength of the E-HAT II software was the reformatting of the scoring to the bottom of the activity page. This re-design allows for the character boxes to be moved up, resulting in more surface area for participants to complete the handwriting tasks. Based on observations, participants had greater ease completely the handwriting tasks and appeared to experience decreased frustration with the task, which is contributed to the reformatting of the scoring, that allows for a larger surface area in which the patients can write.

The fifth strength noted was the E-HAT II's time efficiency. In comparison to the time required to administer all three portions of the Beery VMI, the E-HAT II required only one-half of the amount of time for the VMI. In addition, one clinician based the E-HAT II's clinical utility on the minimal amount of time it required to assess patient handwriting abilities compared to the current pencil/paper assessment tools, which serves to enhance the E-HAT II's marketability within a clinical setting where time demands are prevalent.

Lastly, the newly incorporated ‘Maze’ subtest allowed for motor control to be assessed beyond the domains of handwriting. There are various components that need to be addressed during handwriting rehabilitation, including visual perception, attention, visual-motor integration, motor coordination and fine motor control. (Feder & Majnemer, 2007; Ziviani & Wallen, 2006). This subtest allows for additional components to be incorporated into a writing sample, beyond a more automatic writing task. This addition to the E-HAT II may prove to be an effective test to assess potential impairments beyond the realm of handwriting, in an efficient and objective manner.

In summation, this case study identifies various strengths of the E-HAT II. Thus including, instant feedback and motivation to enhance patient confidence and motivation, improved objectivity of scoring to enhance accuracy, time efficiency to address time constraints in the clinic, and the establishment of an effective means to assess motor control beyond handwriting through the ‘Maze’ subtest.

### Example #4

The E-HAT is an objective assessment that utilizes motion capture technology to measure fine motor control through the task of handwriting. The E-HAT is a software application written in C# programming language for a Windows-based operating system. The tool has undergone multiple pilot and case studies which helped to improve aspects of the E-HAT, like scoring and menu options, and receive feedback on the utility of the assessment. The purpose of the current research was to continue the development process by further assessing the reliability and validity measures of the E-HAT. Particularly, the purpose of this study was to provide additional test scores from pediatric participants, who often experience more variability in fine motor control during a writing task, to the ongoing reliability and validity research of the E-HAT.

### Participants

The study utilized a convenience sampling technique. The participants were recruited through an email invitation to faculty and staff, who have children, at a small, comprehensive college. Participants ranged in age from 5-17 years. Inclusion criteria for the participants included the following: participants will have their dominant hand ready for functional assessment (i.e. no restrictions to the upper extremity or splints, casts, slings); English must be the participants' primary language; participants must have received some formal handwriting education. The invitation yielded 8 pediatric participants which were added to the 50 participants gathered the previous year (see Table 7).

TABLE 7
Demographic Information

| | |
|---|---|
| Age | Range 5-75 years |
| | (17% between 5-17 years) |
| Gender | 83% Female (N = 48) |
| Hand Dominance | 83% Right-handed (N = 48) |

### Instruments

The E-HAT is a software program for a tablet computer that is written in C# programming language for a Windows operating system. The E-HAT provides an objective measure for fine motor control through the task of handwriting. It utilizes uppercase, lowercase, and cursive English letters and Greek and Japanese characters to copy and trace. The assessment portion of the E-HAT also includes an image of a maze to trace. After the assessment is completed, two objective scores are obtained: an average of pixel to pixel accuracy and an average speed.

The pixel to pixel accuracy score is acquired by a scoring algorithm that compares the pixels between the target (known as the letter) and the drawn image (known as the trace). In the scoring summary, the percent of pixel to pixel overlap is the exact overlap between the letter and the trace. A better score is awarded for marks which overlap the target while a lower score is awarded for stray marks and distance away from the letter. Although the average scores are provided at the end of the assessment, the data can be accessed from a separate file to reference later.

### The Beery-Buktenica Developmental Test of Visual-Motor Integration, Sixth Edition (Beery VMI)

The Beery VMI is a standardized paper and pencil assessment designed to evaluate visual and motor abilities through the copying of geometric forms (Beery & Beery, 2010). The Beery VMI full form was used as it was determined to have the best fit to the E-HAT. Scores gathered were checked by an expert scorer, an occupational therapist who has had experience in both administration and scoring of the Beery VMI, to ensure accuracy.

### Procedures

The researchers obtained approval for this study through the Institutional Review Board at Elizabethtown College. The participants were gathered using the outline mentioned previously. Parents and guardians gave consent for their child to participate within the research study, and assent was gained from the child prior to the start of each session. Each child was then given a unique identifying number to separate his or her name from the data collected. Following these initial tasks, the E-HAT was administered which was followed immediately by the administration of the Beery VMI. A short interview was conducted at the end to assess the child's impressions and ease of use of the E-HAT. Each session ranged from 30-45 minutes to complete.

### Data Analysis

Data was analyzed using the Statistical Package for the Social Sciences 19© (SPSS). The statistical analysis included calculating internal consistency using Cronbach's alpha (α) and concurrent validity using a Pearson correlation (r).

### Results

Descriptive statistics were found using the scores from the E-HAT. The mean score on the E-HAT was 55 out of a possible one hundred. The scores ranged from 29-71 out of one hundred with 94% of participants falling within the range of 38-67 points.

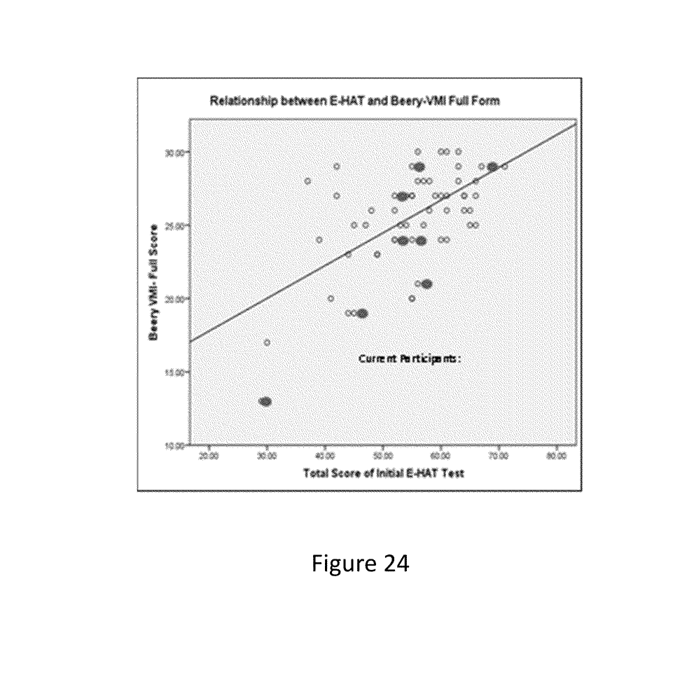

Internal consistency was measured using Cronbach's alpha, which indicated an excellent internal consistency (α=0.91). The analysis of concurrent criterion validity was completed using a Pearson correlation (r). The total score of the E-HAT was compared to the total score of the Beery VMI Full Form. A moderate concurrent validity (r=0.59) was found with the comparison (FIG. 24).

The researchers also gathered qualitative data using the following open-ended questions: “Is there anything you like about the E-HAT?”, “Is there anything you do not like about the E-HAT?”, and How would you change the E-HAT to make it better?” Participant #301 best summarized the group's reactions to using the E-HAT by stating “I like that I can use the pen to touch the computer.” Many suggestions were made on how to improve the E-HAT to make it more approachable to children. Participant #301 also identified “I would like more colors, like purple.” Participant #302 would have liked “if the letters were all in cursive.” Some technological problems were also acknowledged. Participant #304 thought it would have been easier to write if “the computer was thinner” while participant #306 found “it was hard to trace, because [the cursor] moved around a lot.”

### Discussion

Previous research yielded 50 participants and from the data gathered, the results supported that the E-HAT had excellent internal consistency and test retest reliability and moderate concurrent validity. However, the majority of those participants (n=46) possessed similar and developed or mature skillsets. The purpose of the current research was to further assess reliability and validity measures through the addition of participants with a variety of performance skills. Children were selected as the added participants as they are not fluent in handwriting until the ages of 8-9 and the speed of handwriting does not compare to that of an adult until 15 years of age (Graham, Berniger, Weintraub, & Shafer, 1998). The Beery VMI (2010) manual's standard score chart also shows that people, ages 18-50, tend to score the highest, on average. People who are below that age range do not have fully-developed visual motor integration skills while people who are older show decreased abilities in visual motor integration.

The results have shown that the E-HAT has excellent internal consistency and moderate concurrent validity with the Beery VMI Full Form. Excellent internal consistency within the E-HAT supports that the tool is consistently measuring the same construct, which is fine motor control. This supports the E-HAT's reliability. A moderate concurrent validity was reported and expected, as the Beery VMI Full Form and the E-HAT do not measure the exact same construct. The Beery VMI is an assessment of handwriting, but the E-HAT is a measure of fine motor control through the task of handwriting. The inability to achieve a higher level of validity is limiting to the development of the E-HAT. Future researchers should consider utilizing an assessment that looks closely at fine motor control and the upper extremity to maintain a higher level of concurrent validity. For example, the Motor Assessment Scale (MAS) is a standardized assessment that quantitatively measures motor recovery. The items within the test assess upper-arm function, hand movements, and hand activities (Asher, 2007).

A secondary goal of the study was to address the utility of the E-HAT with the pediatric population through an open-ended interview. The eight children provided qualitative feedback about the tool. The results of the interview suggest that the E-HAT could be changed to become more child-friendly. Creating pictures that are age appropriate and more colors are changes that could achieve an assessment that is more relative to children. Development of a nonslip, writing surface, even larger fonts, and a thinner writing surface can also increase the successfulness of not only pediatric users, but also other users who have difficulty with fine motor control. These changes will facilitate the utility users sense when performing the E-HAT.

### Implications for Practice

This study is important to occupational therapy, because it supports the development of an objective, fine motor assessment which is easy to administer, uses minimal effort to score, and is low cost. E-HAT fulfills a need for therapists as there are few objective assessments that measure fine motor control currently used in practice. The E-HAT can be used with a variety of populations (e.g. pediatrics, individuals with neurological impairments, and orthopedics) and in numerous settings (e.g. outpatient clinics, schools, rehabilitation hospitals).

### CONCLUSION

Patients with orthopedic, neurological, or motor problems need to be assessed upon entering occupational services. Objective data that is collected from these assessments must be reliable and valid to provide accurate information to assess intervention strategies and patient results. Through analyzing the psychometric properties of the Electronic-Hand Assessment Tool (E-HAT), the E-HAT can be used as a precise, objective measurement tool of fine motor control.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An electronic hand assessment tool, comprising:
a computing device having a visual display;
a stylus; and
a software application executable on the computing device, such that the software application presents a graphical user interface on the visual display;
wherein the software application presents at least one handwriting exercise to be performed by a subject contacting the stylus to the visual display.

2. The electronic hand assessment tool of claim 1, wherein the at least one handwriting exercise is selected from the group consisting of a trace, a copy and a maze.

3. The electronic hand assessment tool of claim 2, further comprising a scoring system for scoring the performance of the at least one handwriting exercise.

4. The electronic hand assessment tool of claim 3, wherein the scoring system is based on pixel-by-pixel accuracy.

5. The electronic hand assessment tool of claim 3, wherein the scoring system is based on speed of writing.

6. The electronic hand assessment tool of claim 3, wherein the scoring system is based on speed of writing and pixel-by-pixel accuracy.

7. The electronic hand assessment tool of claim 3, wherein the scoring system compares pixels between a target and a user drawn image.

8. The electronic hand assessment tool of claim 7, wherein a score value is awarded on a graded continuum that allots full value points for marks of the user drawn image overlapping the target and a lower score for stray marks of the user drawn image with progressive decrease in score with increasing distance of marks of the user drawn image from the target.

9. The electronic hand assessment tool of claim 7, wherein the scoring system is based on a pixel overlap ratio.

10. The electronic hand assessment tool of claim 9, wherein a score value is based on the amount of the target that is overlapped, how much of the user drawn image is written outside of the lines of the target, and the percentage of how much of the target is colored in by the user drawn image.

11. The electronic hand assessment tool of claim 3, wherein the scoring system comprises a plurality of difficulty settings.

12. The electronic hand assessment tool of claim 3, further comprising an assessment mode.

13. The electronic hand assessment tool of claim 3, further comprising an intervention mode.

14. A method of measuring fine motor control, comprising:
obtaining at least one electronic handwriting exercise on a visual display of a computing device;
performing the at least one electronic handwriting exercise by contacting a stylus to the visual display; and
measuring fine motor control based on the performance of the at least one handwriting exercise.

15. The method of claim 14, wherein the at least one handwriting exercise is selected from the group consisting of a trace, a copy and a maze.

16. The method of claim 15, further comprising scoring the performance of the at least one handwriting exercise.

17. The method of claim 16, wherein the scoring is based on pixel-by-pixel accuracy.

18. The method of claim 16, wherein the scoring is based on speed of writing.

19. The method of claim 16, wherein the scoring is based on speed of writing and pixel-by-pixel accuracy.

20. The method of claim 16, wherein the scoring further comprising comparing pixels between a target and a user drawn image.
